(19) United States
(12) Patent Application Publication
(10) Pub. No.: US 2008/0188791 A1
(43) Pub. Date: Aug. 7, 2008

(54) Active iontophoresis delivery system

(76) Inventors: Attilio E. DiFiore, Taylorsville, UT (US); Jamal S. Yanaki, Salt Lake City, UT (US)

Correspondence Address: KENT S. BURNINGHAM;KENT S. BURNINGHAM, P.C., 466 South 500 East, Salt Lake City, UT, 84102, US

(21) Appl. No.: 11/701,749

(22) Filed: Feb. 2, 2007

(51) Int. Cl. A61N 1/30

(52) U.S. Cl. 604/20

(57) ABSTRACT

A fully integrated, independently accurately performing, active transdermal medicament patch includes a flexible substrate with an adhesive-coated therapeutic face carrying a medicament reservoir. A circuit non-removably carried on the substrate and driven by a light-weight power source causes a substantially constant current to flow for a predetermined therapy period through the reservoir and the skin against which the therapeutic face is disposed. Net electrical polarity of the medicament determines the interconnection of circuit and power source to skin. The circuit includes a field effect transistor or an operational amplifier with a zener diode or a voltage regulator. A timer non-removably carried on the substrate disables the circuit following functioning the therapy period. An active transdermal medicament delivery system employs a single patch or pair of patches physically and electrically connected by a distensible tether.

### RELATED APPLICATIONS

This application is related to U.S. Design Pat. application No. 29/261,600 that was filed on Jun. 16, 2006, and that issued on ______ as U.S. Design Pat. No. ______ for a design titled “Adhesive Transdermal Medicament Patch.”

### BACKGROUND

1. Field of the Invention

The invention disclosed herein relates to the transdermal administration of medicaments to human and animal subjects. More particularly, the present invention pertains to active iontophoretic delivery systems in which electrical contacts are applied to the surface of the skin of a subject for the purpose of delivering medicament through the surface of the skin into underlying tissues.

2. Background Art

During active iontophoresis, direct electrical current is used to cause soluble medicament ions to move across the surface of the skin and to diffuse into underlying tissue. The surface of the skin is not broken by the administration of the medicament. When conducted within appropriate parameters, the sensations experienced by a subject during the delivery of a medicament in this manner are not unpleasant. Therefore, active iontophoresis presents an attractive alternative to hypodermic injections and to intravascular catheterization.

The direct current employed in active iontophoresis systems may be obtained from a variety of electrical power sources, including electrical equipment that ultimately receives power from a wall socket. These power sources are of such bulk, weight, and cost as to necessitate being configured as items of equipment distinct from the electrical contacts that are applied directly to the skin in administering a medicament iontophoretically. Accordingly, such power sources limit the mobility of the patient during the time that treatment is in progress.

In some instances, direct current for an active iontophoretic system is produced by paired regions of contrasting galvanic materials. When coupled by a fluid medium, contrasting galvanic materials produce minute electrical currents that are useful in active iontophoresis. Commensurate with the small size of the currents required, regions of contrasting galvanic materials in active iontophoretic delivery systems are very insubstantial and are usually completely consumed in causing a single administration of medicament. Regions of contrasting galvanic materials are, therefore, printed as thin metallic layers on disposable adhesive patches that are used in many active iontophoretic systems to retain an electrical contact or a reservoir of medicament against the skin of the subject.

A flow of electrical current requires an uninterrupted, electrically-conductive pathway from the positive pole of a power source to the other, negative pole thereof. Living tissue is made up primarily of fluid and is, therefore, a conductor of electrical current. In an iontophoretic circuit, the opposite poles of a power source are electrically coupled to respective, separated contact locations on the skin of the subject. The difference in electrical potential created by the power source between those contact locations causes a movement of electrons and electrically charged molecules, or ions, through the tissue between the contact locations.

The polarity of the net overall electrical charge on dissolved molecules of a medicament determines the contact location at which a supply of the medicament of an active iontophoretic delivery system must be positioned. A positively charged medicament in a reservoir against the skin of a patient must be coupled to the positive pole of any power source that is to be used to administer the medicament iontophoretically. Correspondingly, a reservoir on the skin of a patient containing a negatively charged medicament must be coupled to the negative pole of such a power source. Examples of common iontophoretically administrable medicaments in each category of polarity are listed in the table below.

| Positive Polarity Medicaments | Negative Polarity Medicaments |
|---|---|
| Bupivacaine hydrochloride | Acetic acid |
| Calcium chloride | Betamethasone sodium phosphate |
| Lidocaine hydrochloride | Copper sulfate |
| Zinc chloride | Dexamethasone sodium phosphate |
| Lidocaine | Fentinol |
| | Magnesium sulfate |
| | Naproxen sodium |
| | Sodium chloride |
| | Sodium salicylate |

The medicament supply is housed in a fluid reservoir, which is positioned electrically conductively engaging the skin of the subject at the appropriate of the contact locations. The medicament reservoir can take the forms of a gel suspension of the medicament or a pad of gauze or cotton saturated with fluid containing the medicament. An iontophoretic circuit for driving the medicament through the unbroken skin is established by coupling the appropriate pole of the power source through the medicament reservoir to that contact location and coupling the other pole of the power source to an electrical contact at a location on the skin of the patient distanced from the medicament reservoir.

The medicament reservoir may be conveniently retained against the skin by a first adhesive patch, while the electrical contact at the location distanced from the medicament reservoir may be retained there using a distinct second adhesive patch. Alternatively, both the medicament reservoir and the electrical contact for the location distanced from the medicament reservoir may be carried on a single adhesive patch at respective electrically isolated locations.

The use of iontophoresis to administer medicaments to a subject is advantageous in several respects.

Medications delivered by an active iontophoretic system bypass the digestive system. This reduces digestive tract irritation. In many cases, medicaments administered orally are less potent than if administered transcutaneously. In compensation, it is often necessary in achieving a target effective dosage level to administer orally larger quantities of medicament than would be administered transcutaneously.

Active iontophoretic systems do not require intensive skin site sanitation to avoid infections. Patches and the other equipment used in active iontophoresis do not interact with bodily fluids and, accordingly, need not be disposed as hazardous biological materials following use. Being a noninvasive procedure, the administration of medicament with an active iontophoretic system does not necessitate tissue injury, as is the case with hypodermic injections and with intravenous catheterizations. Needle punctures and catheter implantations inherently involve the experience of some degree of pain. Repeated needle punctures in a single anatomical region and long term catheter residence can adversely affect the health of surrounding tissue. These unintended consequences of invasive transcutaneous medicament administration are particularly undesirable in an injured area of the body that is to be treated directly with medicament, such as the in the treatment of strained muscles or tendons.

With some exceptions, no pharmacologically significant portion of a medication delivered iontophoretically becomes systemically distributed. Rather, a medication delivered iontophoretically remains localized in the tissue at the site of administration. This minimizes unwanted systemic side effects, reduces required dosages, and lightens the burdens imposed on the liver and kidneys during metabolization of the medication.

The dosage of a medicament delivered iontophoretically is conveniently and accurately measured by monitoring the amount and the duration of the current flowing during the administration. With current being measured in amperes and time being measured in minutes, the dosage of medicament given transcutaneously is given in units of ampere-minutes. Due to the minute quantities of medicament required in active iontophoresis, medicament dosage in active iontophoresis is generally prescribed in milliamp-minutes. Dosage measured in this manner is more precise than is dosage measures as a fluid volume or as a numbers of tablets.

Finally, the simplicity of active iontophoretic equipment does not require the skills of nurses or doctors. This favors convenience and reduces the costs associated with medicament delivery.

### SUMMARY OF THE INVENTION

The present invention promotes the wide use of active iontophoretic systems by providing improved components for active iontophoretic systems. The present invention thus improves the safety of patients and medical personnel.

The teachings of the present invention enhance the reliability and simplicity of active iontophoretic systems and lead to a reduction in costs associated with the manufacture of such systems, as well as with the use of such systems to deliver medication.

In one aspect of the present invention a fully integrated, independently accurately performing adhesive active transdermal medicament patch is provided. Another aspect of the present invention implements such teachings in a system utilizing a plurality of adhesive patches.

The present invention contemplates related methods of manufacture and related methods of patient treatment.

Additional objects and advantages of the invention will be set forth in the description which follows, and in part will be obvious from the description, or may be learned by the practice of the invention. The objects and advantages of the invention may be realized and obtained by means of the instruments and combinations particularly pointed out in the appended claims.

### BRIEF DESCRIPTION OF THE DRAWINGS

The manner in which the above-recited and other advantages and objects of the invention are obtained will be understood by a more particular description of the invention rendered by reference to specific embodiments thereof that are illustrated in the appended drawings. These figures are intended to be illustrative, not limiting. Although the invention is generally described in the context of these embodiments, it should be understood that it is not intended to limit the scope of the invention to these particular embodiments.

Understanding that these drawings depict only typical embodiments of the invention and are not therefore to be considered limiting of its scope, the invention will be described and explained with additional specificity and detail through the use of the accompanying drawings in which:

FIG. 1 is a perspective view of a first embodiment of an active iontophoretic delivery system incorporating teachings of the present invention being worn by a patient requiring the localized administration of a medicament;

FIG. 2 is a perspective view of an active transdermal patch suitable for use in iontophoretic delivery system of FIG. 1 showing a release liner in the process of being peeled from an adhesive coating on the therapeutic face of that patch;

FIG. 3 is a perspective view of the therapeutic face of the active transdermal patch of FIG. 2 with the release liner shown in FIG. 2 fully removed;

FIG. 4 is a perspective view of the face of the active transdermal patch of FIGS. 2 and 3 on the opposite side of that patch from the side shown in FIGS. 2 and 3;

FIG. 5 is a cross-sectional elevation view of the active transdermal patch of FIG. 4 taken along section line 5-5 shown therein;

FIG. 6A is a diagram illustrating the movement of a medicament of positive polarity in the skin of a wearer of the active transdermal patch of FIGS. 2-5;

FIG. 6B is a diagram illustrating the movement of a medicament of negative polarity in the skin of a wearer of the active transdermal patch of FIGS. 2-5;

FIG. 7 is a schematic drawing of a first embodiment of electronics embodying teachings of the present invention and suitable for use on the active transdermal patch of FIGS. 2-5;

FIG. 8 is a schematic drawing of a second embodiment of electronics embodying teachings of the present invention and suitable for use on the active transdermal patch of FIGS. 2-5;

FIG. 9 is a schematic drawing of a third embodiment of electronics embodying teachings of the present invention and suitable for use on the active transdermal patch of FIGS. 2-5;

FIG. 10 is a graph depicting the performance of the electronics of FIG. 9 across a physiologically relevant, or meaningful, range of conductive skin resistances in a patient likely to receive an administration of a medicament using the active transdermal patch of FIGS. 2-5;

FIG. 11 is a schematic drawing of a fourth embodiment of electronics embodying teachings of the present invention and suitable for use on the active transdermal patch of FIGS. 2-5;

FIG. 12 is a schematic drawing of a fifth embodiment of electronics embodying teachings of the present invention and suitable for use on the active transdermal patch of FIGS. 2-5;

FIG. 13 is a schematic drawing of a sixth embodiment of an electrical circuit embodying teachings of the present invention and suitable for use on the active transdermal patch of FIGS. 2-5;

FIGS. 14A-14F are related graphs depicting comparatively the performance of the electronics of FIG. 13 for various levels of input power supply and various therapy periods across meaningful ranges of likely conductive skin resistance in a patient using the active transdermal patch of FIGS. 2-5;

FIG. 15 is a schematic drawing of a seventh embodiment of electronics embodying teachings of the present invention and suitable for use on the active transdermal patch of FIGS. 2-5;

FIG. 16 is a perspective view of a second embodiment of an active iontophoretic delivery system incorporating teachings of the present invention being worn by a patient requiring the localized administration of a medicament;

FIG. 17 is a perspective view of a pair of active transdermal patches suitable for use in iontophoretic delivery system of FIG. 16 showing release liners in the process of being peeled from an adhesive coating on the therapeutic faces of those patches;

FIG. 18 is a perspective view of the therapeutic faces of the active transdermal patches of FIG. 17 with the release liners shown in FIG. 17 fully removed;

FIG. 19 is a perspective view of the faces of the active transdermal patches of FIGS. 17 and 18 on the opposite side of those patches from the sides shown in FIGS. 17 and 18;

FIG. 20 is a cross-sectional elevation view of the active transdermal patches of FIG. 19 taken along section line 20-20 shown therein;

FIG. 21A is a diagram illustrating the movement of a medicament of positive polarity in the skin of a wearer of the active transdermal patch of FIGS. 16-20; and

FIG. 21B is a diagram illustrating the movement of a medicament of negative polarity in the skin of a wearer of the active transdermal patch of FIGS. 16-20.

### DETAILED DESCRIPTION OF THE INVENTION

In the following description, for purpose of explanation, specific details are set forth in order to provide an understanding of the invention. It will be apparent, however, that the invention may be practiced without these details. It will also be recognized that embodiments of the present invention, some of which are described below, may be incorporated into a number of different electrical components, circuits, devices, and systems. Structures and devices shown in block diagram are illustrative of exemplary embodiments of the invention and are intended to simplify discussion of the teachings of the present invention, thereby to avoid obscuring the invention. Furthermore, connections between components within the figures are not intended to be limited to direct connections. Rather, connections between such components may be modified, reformatted, or otherwise changed to include intermediary components.

Reference in the specification to “one embodiment” or “an embodiment” means that a particular feature, structure, characteristic, or function described in connection with the embodiment is included in at least one embodiment of the invention. The use of the phrase “in one embodiment” in various places in the specification does not necessarily refer to any single embodiment of the invention.

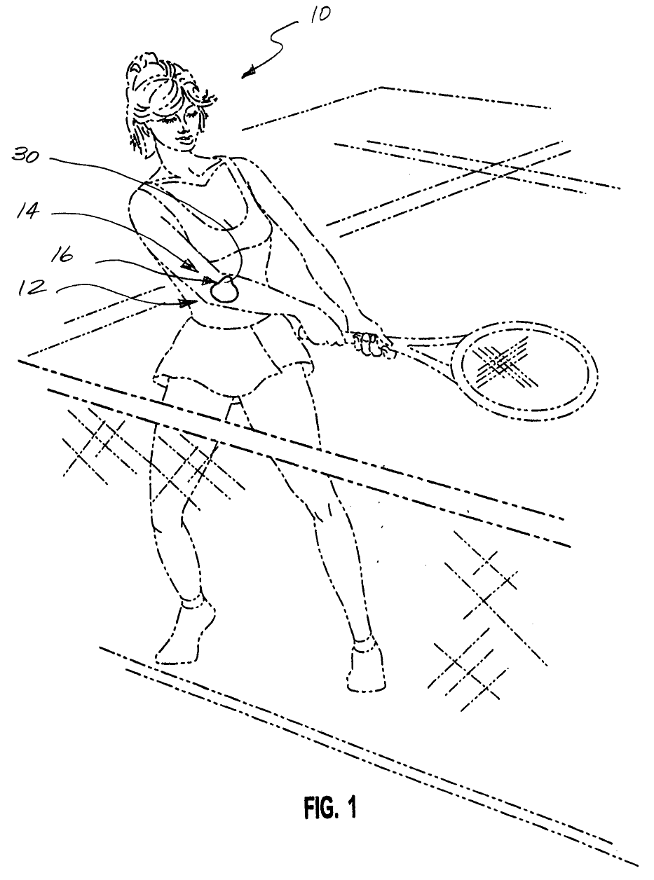

FIG. 1 shows a patient 10 requiring the localized administration of a medicament to elbow 12 thereof. For that purpose, patient 10 is wearing on elbow 12 thereof a first embodiment of an active iontophoretic delivery system 14 that incorporates teachings of the present invention. While so doing, patient 10 is nonetheless able to engage in vigorous physical activity, because delivery system 14 is entirely self-contained, and not supplied with power from any immobile or cumbersome power source. Delivery system 14 takes the form of an active transdermal medicament patch 16 that is removable adhered to the skin of elbow 12 of patient 10 for the duration of a predetermined therapy period. The length of the therapy period during which patch 16 must be worn is determined by the rate at which patch 16 delivers medicament through the skin of patient 10 and the total dose of medicament that is to be administered.

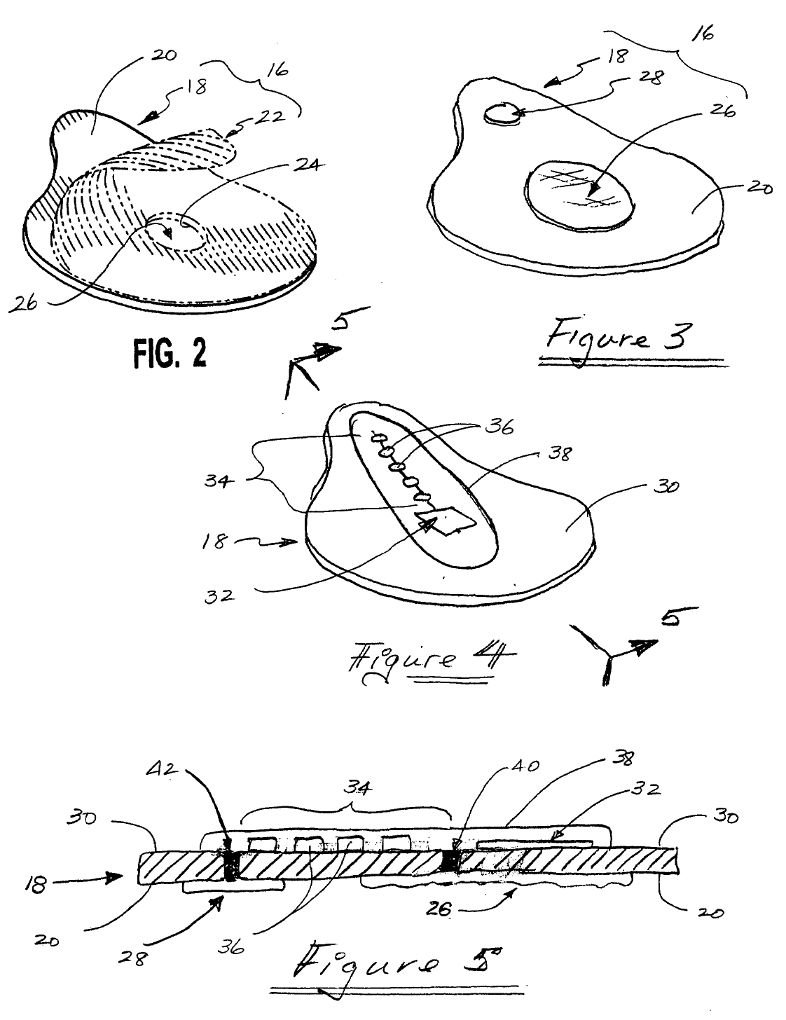

FIGS. 2-5 taken together afford an overview of the structural elements of patch 16.

FIG. 2 reveals that patch 16 includes a flexible, planar biocompatible substrate 18 having a therapeutic face 20 on one side thereof that is intended to be disposed in contact with the skin of a patient, such as patient 10 in FIG. 1. Therapeutic face 20 is coated with a biocompatible adhesive and is for that reason removable securable to the skin of patient 10. Prior to the actual use of patch 16, the adhesive on therapeutic face 20 is shielded by a removable release liner 22, which is shown in FIG. 2 in the process of being peeled away from therapeutic face 20. Formed centrally through release liner 22 is a medicament aperture 24. The function of medicament aperture 24 will be explained below.

FIG. 3 shows therapeutic face 20 of patch 16 after the complete removal of release liner 22 therefrom. There it can bee seen that patch 16 includes a medicament reservoir 26 that is positioned on therapeutic face 20 of substrate 18 interior of the periphery of therapeutic face 20. Reservoir 26 is intended to electrically conductively engage the skin of patient 10, when therapeutic face 20 of substrate 18 is disposed against the skin of patient 10. Reservoir 26 can take the form of a gel suspension of medicament or a pad of gauze or cotton saturated with fluid containing medicament. It is the purpose of medicament aperture 24 in release sheet 22 to permit the disposition of medicament in reservoir 26 prior to the removal of release sheet 22 from therapeutic face 20 of substrate 18.

An electrical contact 28 is also positioned on therapeutic face 20 of substrate 18, but electrical contact 28 is separated from reservoir 26, and thus electrically isolated therefrom. Electrical contact 28 is also capable of electrically conductively engaging the skin of patient 10 when therapeutic face 20 of substrate 18 is disposed against the skin. Accordingly, when as shown in FIG. 1, patch 16 is adhered to the skin of patient 10, electrical contact 28 engages the skin of patient 10 at a location that is remote from reservoir 26.

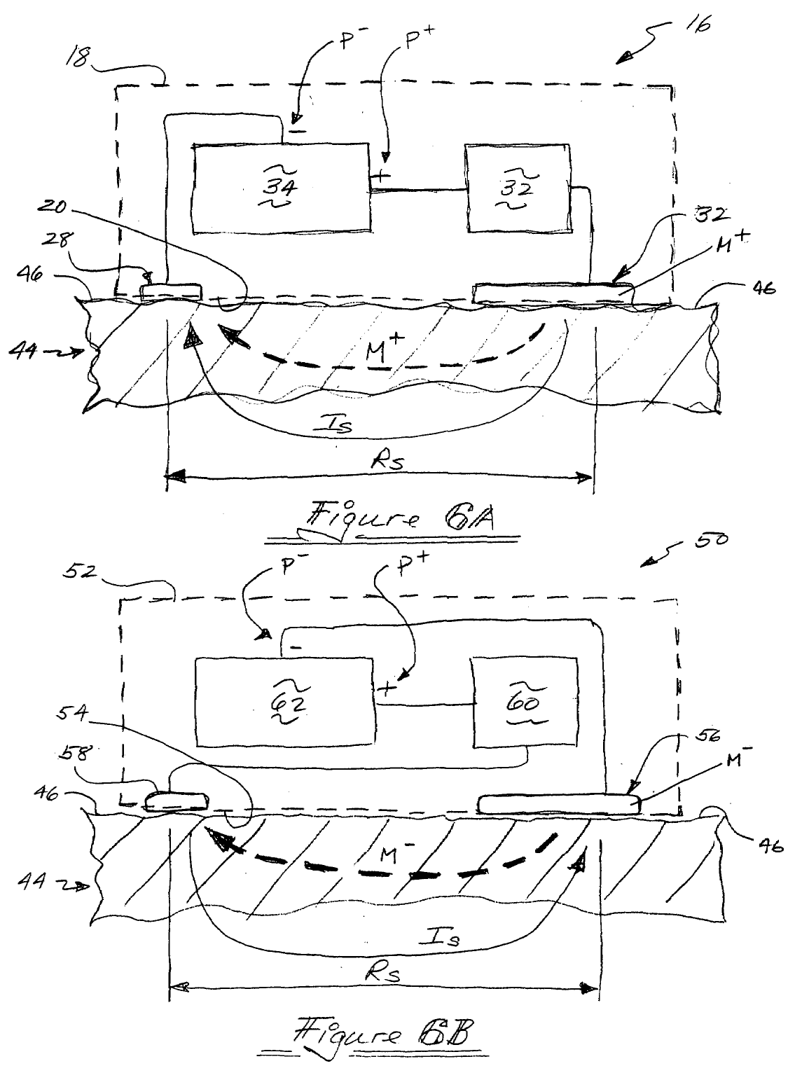

FIG. 4 shows the upper face 30 of substrate 18, which is the face of substrate 18 on the opposite side of substrate 18 from therapeutic face 20 shown in FIGS. 2 and 3. Upper face 30 is thus the face of substrate 18 that is visible when worn by patient 10 in FIG. 1. Upper face 30 of substrate 18 carries electronic circuitry 32 and a corresponding power source 34, which is shown by way of example as being a plurality of series-connected miniature batteries 36, each of about 3 volts potential. Power source 34 thus supplies non-alternating current to electronic circuitry 32. Electronic circuitry 32 and power source 34 are shown as being encased on upper face 30 of substrate 18 by a transparent protective cover 38, but either or both of power source 34 and electronic circuitry 32 could with equal functional adequacy be partially or wholly imbedded in substrate 18, or even carried on therapeutic face 20 thereof.

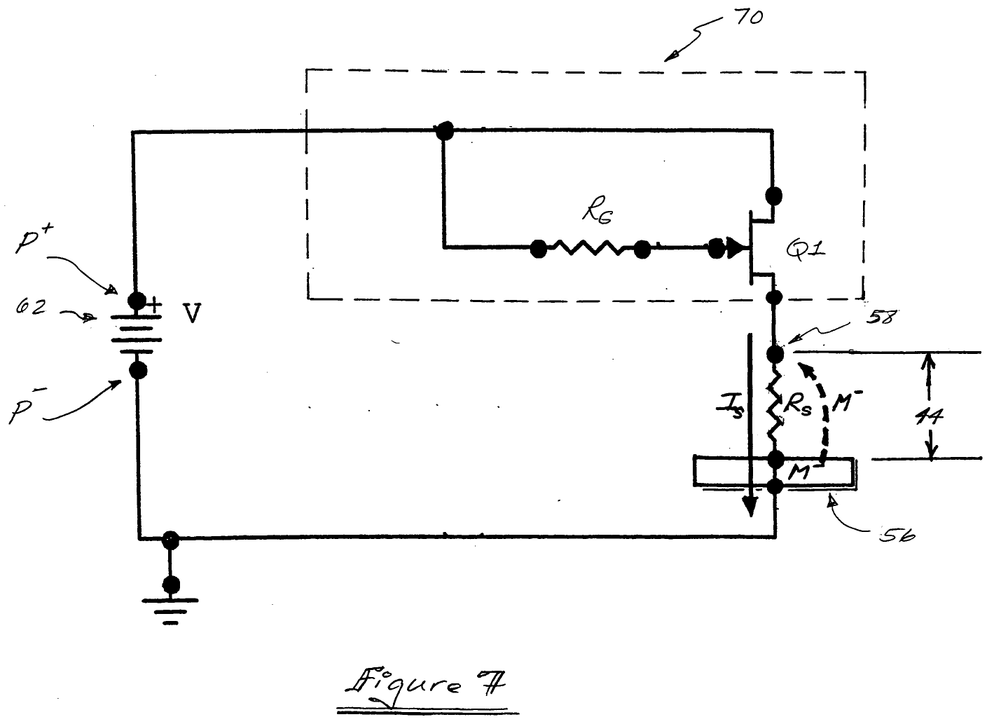

FIG. 5 is an elevation cross section view of patch 16 taken along section line 5-5 in FIG. 4. As a result, FIG. 5 depicts in edge view both sides of substrate 18, as well as the interaction through substrate 18 of the features of patch 16 shown in FIGS. 2-4. Reservoir 26 and electrical contact 28 are shown as being carried on therapeutic face 20 of substrate 18, while electronic circuitry 32 and power source 34 encased in cover 38 are shown carried on upper face 30. Reservoir 26 and electrical contact 28 are electrically isolated from each other. One of electronic circuitry 32 and power source 34 is electrically interconnected by way of a first via 40 through substrate 18 to reservoir 26, while the other of electronic circuitry 32 and power source 34 is electrically interconnected by way of a second via 42 through substrate 18 to electrical contact 28. If either or both of power source 34 and electronic circuitry 32 is partially or wholly imbedded in substrate 18 or carried on therapeutic face 20, the need for one or both of vias 40, 42, may be obviated.

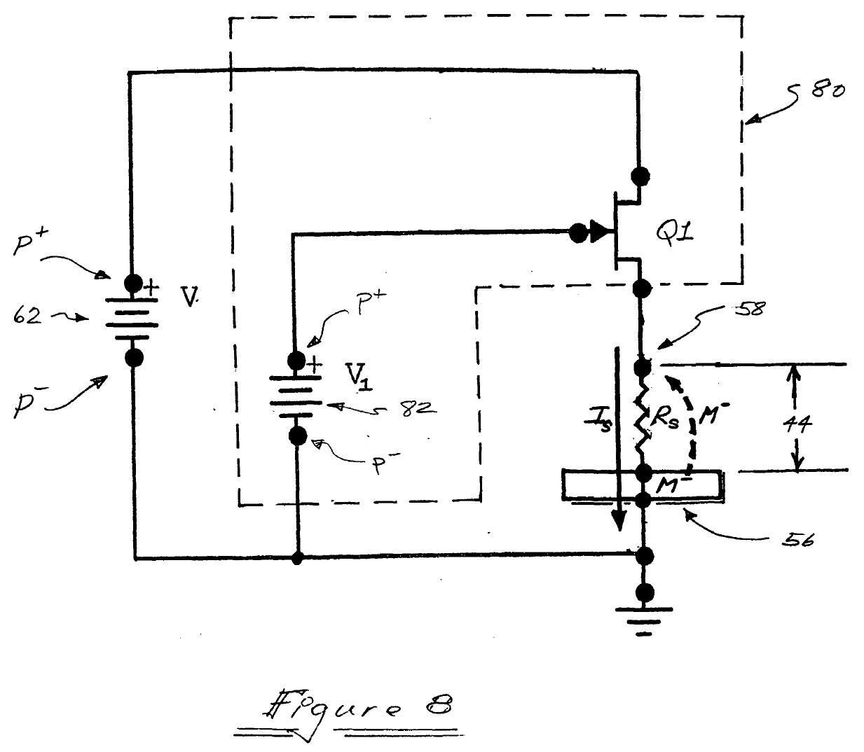

FIGS. 6A and 6B are related diagrams that compare the movement of medicaments of differing polarities through the skin of a wearer of active transdermal patch 16 and the altered electrical interconnections required among element of patch 16 to produce those movements.

FIG. 6A illustrates the movement of molecules of a positive medicament M⁺that exhibits a net positive polarity. Therapeutic face 20 of substrate 18 is shown as being disposed against the surface 46 of skin 44. Then reservoir 26 and electrical contact 28 each electrically conductively engage surface 46 of skin 44 at separated locations. Aside from the conductivity of skin 44, these locations are electrically isolated from each other. The positive pole P⁺ of power source 34 is coupled through electronic circuitry 32 to reservoir 26. The negative pole P⁻ of power source 34 is coupled directly to electrical contact 28, which engages skin 44 at a location remote from reservoir 26. The electromotive differential thusly applied to skin 44 between reservoir 26 and electrical contact 28 induces molecules of positive medicament M⁺ to move as positive ions out of reservoir 26 toward skin 44, across the unbroken surface 46 of skin 44, and through skin 44 in the direction of electrical contact 28. This movement is indicated in FIG. 6A by a dashed arrow labeled M⁺.

In electrical circuits, the flow of current is conventionally indicated as a flow through the circuit from the positive to the negative pole of the power source employed therewith. Therefore, in FIG. 6A, a skin current I_{S} is schematically indicated by a solid arrow to flow through skin 44 from reservoir 26 that is associated with positive pole P⁺ of power source 34 to electrical contact 28 that is associated with negative pole P⁻ of power source 34. In the use of patch 16 to administer a positive medicament M⁺, the direction of movement of molecules of positive medicament M⁺ through skin 44 thus coincides with the direction of electrical current I_{S}.

While living tissue is a conductor of electric current, living tissue does nonetheless resist the flow of electrical current therethrough. It is the function of power source 34 to apply a sufficient electromotive force differential through skin 44 between reservoir 26 and electrical contact 28 as to overcome this resistance. The presence of electrical resistance in skin 44 is indicated schematically in FIG. 6A as skin resistance R_{S}. Skin resistance R_{S} varies among human subjects over a physiological relevant, or meaningful, range of current flow skin resistance in a wide range of from about 20 kilo-ohms to about 70 kilo-ohms. More narrowly, skin resistance R_{S} varies among human subjects in a meaningful range of from about 25 kilo-ohms to about 50 kilo-ohms. Most narrowly, skin resistance R_{S} varies among human subjects in a meaningful range of from about 30 kilo-ohms to about 40 kilo-ohms.

In FIG. 6B, transcutaneous administration is intended of molecules of a negative medicament M⁻ that exhibits a net negative polarity. Under such conditions the electrical components of an active medicament patch incorporating teachings of the present invention must be altered from those shown in FIG. 6A. Accordingly, in FIG. 6B a second embodiment of an active medicament patch 50 incorporating teachings of the present invention is shown to include a substrate 52 having an adhesive-coated therapeutic face 54. On therapeutic face 54 are a medicament reservoir 56 and an electrical contact 58 separated therefrom. Electronic circuitry 60 and a power source 62 are included in patch 50, and reservoir 56 is filled with molecules of a negative medicament M⁻.

Therapeutic face 54 of substrate 52 is shown as being disposed against surface 46 of skin 44. Then reservoir 66 and electrical contact 58 each electrically conductively engage surface 46 of skin 44 at separated locations. Aside from the conductivity of skin 44, these locations are electrically isolated from each other. Positive pole P⁺ of power source 62 is coupled through electronic circuitry 32 to electrical contact 58. Correspondingly, negative pole P of power source 62 is coupled directly to reservoir 56. The electromotive differential thusly applied to skin 44 between electrical contact 58 and reservoir 56 induces molecules of negative medicament M⁻ to move as negative ions out of reservoir 56 toward skin 44, across the unbroken surface 46 of skin 44, and through skin 44 in the direction of electrical contact 58. This movement is indicated in FIG. 6B by a dashed arrow labeled M⁻.

The flow of current in an electrical circuit is conventionally indicated as a flow through the circuit from the positive to the negative pole of the power source employed therewith. In FIG. 6B, a skin current I_{S} is schematically indicated by a solid arrow to flow through skin 44 toward reservoir 56, which is associated with negative pole P⁻ of power source 62, from electrical contact 58, which is associated through electronic circuitry 60 with positive pole P⁺ of power source 62. In the use of patch 60 to administer negative medicament M⁻, the movement of molecules of negative medicament M⁻ through skin 44 is in a direction that is opposite to that of electrical current I_{S}. The presence of electrical resistance in skin 44 is indicated schematically as skin resistance R_{S}.

For convenience and consistency in discussing the various embodiments of the invention that are to be disclosed subsequently, the convention will be uniformly observed that a negative medicament is to be administered. Nonetheless, this is not an indication that the teachings of the present invention have relevance exclusively to the administration of negative medicaments, as the present invention has applicability with equal efficacy to the administration of positive medicaments.

According to one aspect of the present invention, an active transdermal medicament patch, such as patch 16 in FIGS. 2-6A or patch 50 in FIG. 6B, includes current means non-removably carried on the substrate of the patch that is driven by a power source that is also carried on that substrate. The current means is for causing a substantially constant current to flow through the medicament reservoir of the patch and the skin of a wearer of the patch during the entire course of some predetermined therapy period. In this manner, the total dose of medicament delivered by an active transdermal medicament patch incorporating teachings of the present invention is with reasonable medical reliability determinable by reference to the total time during which the patch is employed for therapy.

The absolute accuracy of this manner of measuring the actual dosage of a medicament delivered by the apparatus and methods of the present invention is necessarily qualified to some degree.

At the commencement of the passage of current through the skin of a patient, the resistance of the skin to the passage of current is far higher than is the skin resistance R_{S} once a flow of current has been established. Accordingly, for a few initial minutes of a predetermined therapy period, amounts of current will necessarily flow through the skin that vary somewhat from the stable level of current subsequently observed during the balance of the therapy period. Nonetheless, over a therapy period of a few hours, this initial variation in the amount of current passing through the skin has been determined to have a negligible effect on the overall dose of medicament ultimately administered.

Similarly, certain electrical components of the types called for in various of the examplary embodiments of inventive circuits disclosed herein are occasionally susceptible, due to heating or otherwise, of mildly transient start-up performances. These also stabilize after a relatively short fraction of any normal therapy period and produce no more than a negligible effect on the overall dose of medicament ultimately administered during that entire therapy period.

As a result, it is contemplated that any such biological or electrical transients as might be observable in commencing the administration of medicament using apparatus of the method of the present invention do not derogate from what will generally be rendered to as a substantially constant current flow through the medicament reservoir of the patch and the skin of a wearer of the patch during the entire course of some predetermined therapy period.

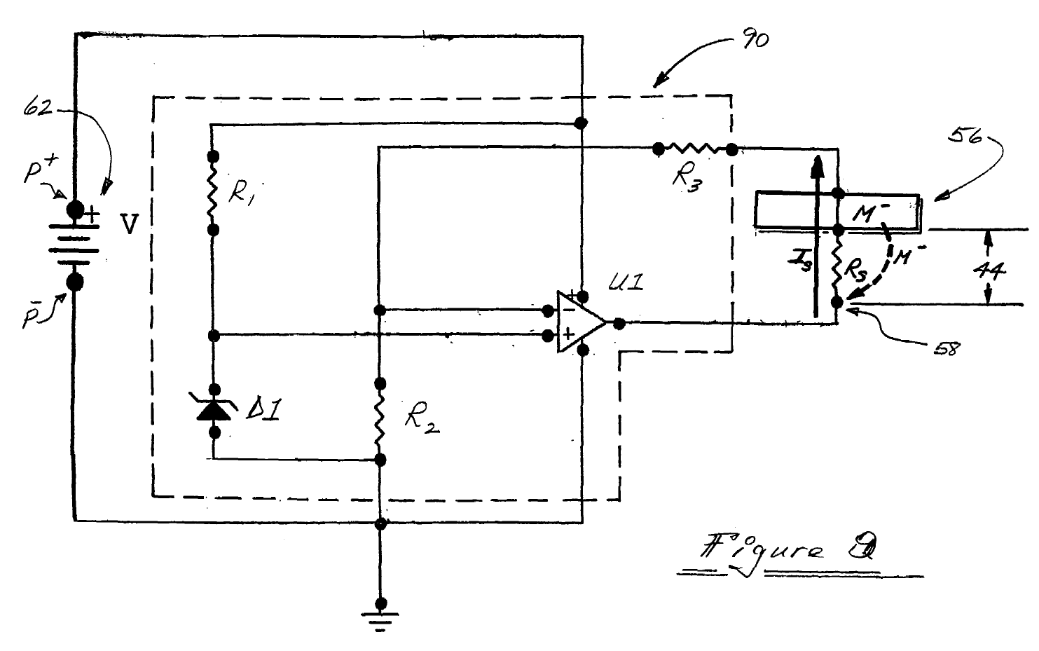

By way of example and not limitation, shown in FIG. 7 is a first embodiment of electronics capable of performing the function of a current means according to teachings of the present invention. There, a circuit 70 is coupled to the positive pole P⁺ of power source 6, which is capable at the outset of supplying a of voltage V for driving circuit 70. Power source 62 causes an electrical current I_{S} to flow through skin 44 of a patient in the direction shown, overcoming in the process electrical skin resistance R_{S} of skin 44. The negative pole P⁻ of power source 62 is coupled to medicament reservoir 56, which is filled, according to the convention set forth above, with molecules of a negative medicament M⁻. As a result, a flow of molecules of medicament M⁻ is induced from reservoir 56, through skin 44, and toward electrical contact 58 in a direction that is opposite to that of electrical current I_{S}.

Circuit 70 is so configured as to cause electrical current I_{S} to be substantially constant for the full duration of a predetermined therapy period in a range of from 5 to about 8 hours, or most commonly from about 6 hours to about 7 hours. Circuit 70 includes a field effect transistor Q1 that is source-to-drain series-connected between positive pole P⁺ of power source 62 and electrical contact 58 on skin 44. The gate of field effect transistor Q1 is coupled through a gate resistor R_{G} to positive pole P⁺ of power source 62. For a skin resistance R_{S}=35 kilo-ohms, the following circuit component values and identities produced a substantially constant electrical current I_{S}=0.341 milliamperes:
Q1=J-type field effect transistor NTE312 of the type manufactured by NTE Electronics;
R_{G}=100 ohms; and
V=12 volts.
   In the case of circuit 70, the duration of therapy is controlled by noting the time at which the patch carrying circuit 70 is first disposed against the surface of the skin of a patient, and then by removing and discarding the patch at the end of the appropriate therapy period. For the circumstances depicted in FIG. 7 and described above, the table that follows sets forth the total dose R_{X} of molecules of medicament M⁻ that would be delivered during several typical therapy periods.

| Therapy Period | Total Dose |
|---|---|
| Duration (hours) | (milliamp-minutes) |
| 5 | 102.3 |
| 6 | 122.8 |
| 7 | 143.2 |
| 8 | 163.7 |

By way of example and not limitation, shown in FIG. 8 is a second embodiment of electronics capable of performing the function of a current means according to teachings of the present invention. There, a circuit 80 is coupled to the positive pole P⁺ of power source 62, which is capable at the outset of supplying a voltage V for driving circuit 80. Power source 62 causes an electrical current I_{S} to flow through skin 44 of a patient in the direction shown, overcoming in the process electrical skin resistance R_{S} of skin 44. The negative pole P⁻ of power source 62 is coupled to medicament reservoir 56, which is filled, according to the convention set forth above, with molecules of a negative medicament M⁻. As a result, a flow of medicament M⁻ is induced from reservoir 56, through skin 44, and toward electrical contact 58 in a direction that is the oppose of that exhibited by electrical current I_{S}.

Circuit 80 is so configured as to cause electrical current I_{S} to be substantially constant for the full duration of a predetermined therapy period in a range of from 5 to about 8 hours, or more commonly from about 6 hours to about 7 hours. Circuit 80 includes a field effect transistor Q1 that is source-to-drain series-connected between positive pole P⁺ of power source 62 and electrical contact 58 on skin 44. The gate of field effect transistor Q1 is held at an invariant voltage level by a gate voltage power source 82. The positive pole P⁺ of gate voltage power source 82 is coupled to the gate of field effect transistor Q1, while the negative pole P⁻ of gate voltage power source 82 is coupled to negative pole P⁻ of power source 62. For a skin resistance R_{S}=35 kilo-ohms, the following circuit component values and identities produced a substantially constant electrical current I_{S}=0.240 milliamperes:
Q1=J-type field effect transistor NTE312 of the type manufactured by NTE Electronics;
V₁=6 volts; and
V=18 volts.

In the case of circuit 80, the duration of therapy is controlled by noting the time at which the patch bearing circuit 80 is first disposed against the surface of the skin of a patient, and then by removing and discarding the patch at the end of the appropriate therapy period. For the circumstances depicted in FIG. 8 and described above, the table that follows sets forth the total dose R_{X} of molecules of medicament M⁻ that would be delivered during several typical therapy periods.

| Therapy Period | Total Dose |
|---|---|
| Duration (hours) | (milliamp-minutes) |
| 5 | 72.0 |
| 6 | 86.4 |
| 7 | 100.8 |
| 8 | 115.2 |

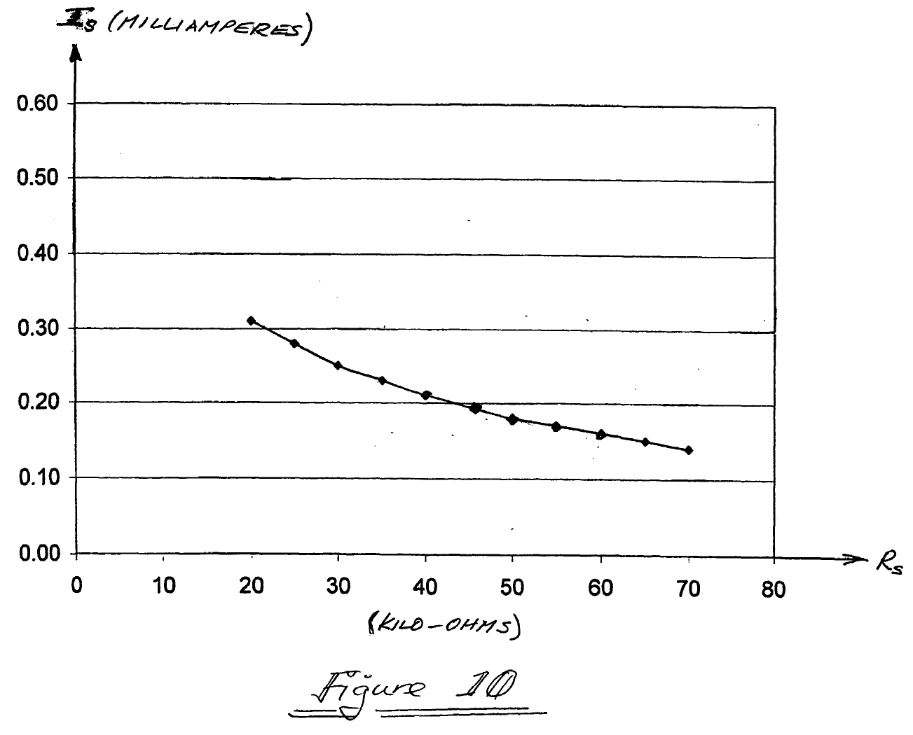

By way of example and not limitation, shown in FIG. 9 is a third embodiment of electronics capable of performing the function of a current means according to teachings of the present invention. There a circuit 90 is coupled to the positive pole P⁺ of power source 62, which is capable at the outset of supplying a voltage V for driving circuit 90. Power source 62 causes an electrical current I_{S} to flow through skin 44 of a patient in the direction shown, overcoming in the process electrical skin resistance R_{S} of skin 44. The negative pole P⁻ of power source 62 is coupled, albeit indirectly, to medicament reservoir 56, which is filled, according to the convention set forth above, with molecules of a negative medicament M⁻. As a result, a flow of molecules of medicament M⁻ is induced from reservoir 56, through skin 44, and toward electrical contact 58 in a direction opposite to that of electrical current I_{S}.

Circuit 90 is so configured as to cause electrical current I_{S} to be substantially constant for the full duration of a predetermined therapy period in a range of from 5 to about 8 hours, or more commonly from about 6 hours to about 7 hours. Circuit 90 includes an operational amplifier U1, a zener diode D1, and various biasing resistors R₁, R₂, and R₃ that are connected as shown. For a skin resistance R_{S}=35 kilo-ohms, the following circuit component values and identities produced a substantially constant electrical current I_{S}=0.227 milliamperes:
U1=operational amplifier UA741 of the type manufactured by Fairchild Semiconductor;
D1=zener diode 1N4733 of the type manufactured by General Semiconductor;
R₁=1 kilo-ohm;
R₂=100 ohms;
R₃=27.4 kilo-ohms; and
V=15 volts.

In the case of circuit 90, the duration of therapy is controlled by noting the time at which the patch carrying circuit 90 is first disposed against the surface of the skin of a patient, and then by removing and discarding the patch at the end of the appropriate therapy period. For the circumstances depicted in FIG. 9 and described above, the table that follows sets forth the total dose R_{X} of molecules of medicament M⁻ that would be delivered for several typical therapy periods.

| Therapy Period | Total Dose |
|---|---|
| Duration (hours) | (milliamp-minutes) |
| 5 | 68.1 |
| 6 | 81.7 |
| 7 | 95.3 |
| 8 | 109.0 |

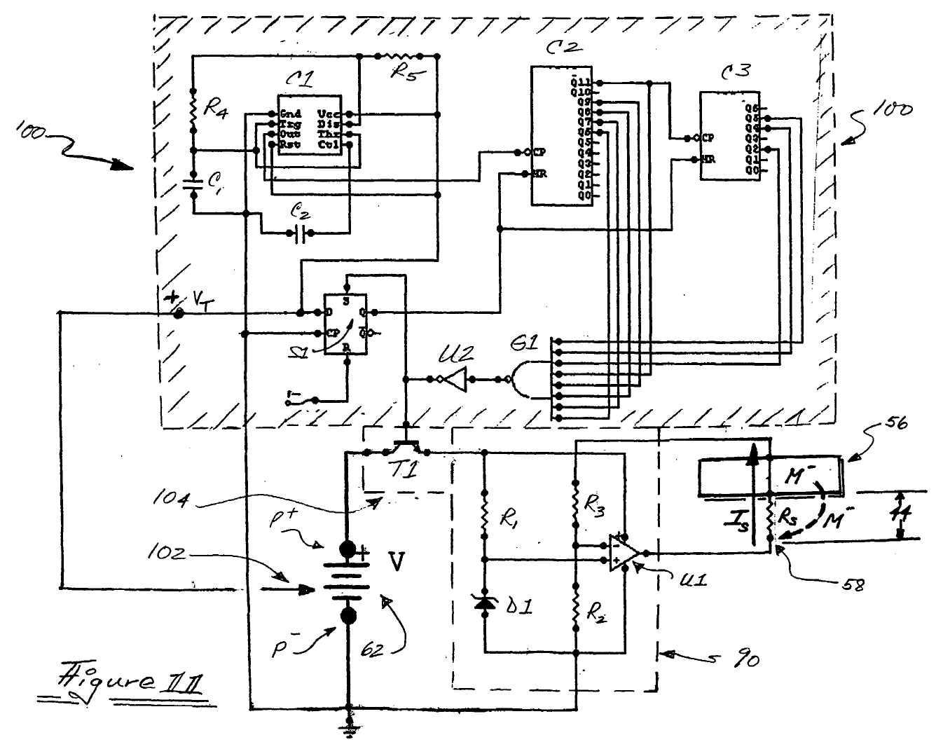

FIG. 10 is a graph depicting the performance of electrical circuit 90 of FIG. 9 across a physiologically relevant, or meaningful, range of conductive skin resistances in patients likely to receive an administration of a medicament using active transdermal patch 16 of FIGS. 2-5. The range of conductive skin resistances over which the performance of circuit 90 was tested was for skin resistance R_{S} in a range from about 20 kilo-ohms to about 70 kilo-ohms. In that range, the value of electrical current I_{S} through skin 44 of any given individual patient was in a range of from about 0.148 milliamperes to about 0.310 milliamperes. A target electrical current I_{S} of about 0.230 milliamperes plus or minus 10 milliamperes extended over a meaningful range of skin resistance R_{S} from about 30 kilo-ohms to about 40 kilo-ohms.

According to yet another aspect of the present invention, an electric circuit, such as circuits 70, 80, or 90, is accompanied on an active iontophoresis patch by control means carried on the substrate of that patch. The control means is for disabling the electric circuit following the operation of that circuit for a predetermined therapy period. Thus, the inclusion in an active iontophoresis patch of a control means according to teachings of the present invention enables the automatic control of the duration of therapy, regardless of the amount of time that the patch is in contact with the skin of a patient. This eliminates any need to note with specificity the time at which the patch is first disposed against the surface of the skin of the patient, or to remove the patch promptly at the end of the chosen therapy period.

As shown in FIG. 11 by way of example and not limitation is a fourth embodiment of electronics embodying teachings of the present invention. There, circuit 90 as described above in relation to FIG. 9 is provided with a timer 100 that performs the functions of a control means according to teachings of the present invention. In the embodiment illustrated, timer 100 is a flip-flop down-counter. Timer 100 includes counters C1, C2, and C3 interconnected as shown. Counter C1 is suitably biased for use in those relationships by resistors R4 and R₅ and capacitors C₁ and C₂ that are connected to counter C1 and to other elements of timer 100 as shown. Output signals from counters C2 and C3 are communicated as input signals to a NAND-gate G1. NAND-gate G1 generates a therapy termination signal when sufficient increments of time that cumulatively equal the chosen therapy period have been accounted for by counters C1, C2, and C3. The therapy termination signal is enhanced in an amplifier U2 and communicated both, internally of timer 100 to a reset switch S1, and externally thereof as an output signal with which to conclude the operation of circuit 90. The components of timer 100 are supplied with power at an electromotive force differential of V_{T} that is derived by way of a tap 102 on power source 62.

Accordingly, by way of example, the enhanced therapy termination signal from timer 100 is used to operate a switch 104 positioned between power source 62 and circuit 90. Switch 104 includes a transistor T1 that is source-to-drain series-connected between positive pole P⁺ of power source 62 and operational amplifier U1 in circuit 90. The gate of transistor T1 is supplied with any therapy termination signal generated by timer 100. In response, transistor T1 becomes non-conducing, and the supply of power to circuit 90 ceases. Correspondingly, electric current I_{S} through skin 44 of a patient is curtailed, along with the corresponding flow of molecules of negative medicament M⁻ from reservoir 56 through skin 44 toward electrical contact 58. The patch carrying circuit 90, timer 100, and switch 104 can then continue to be worn by the patient and removed at a convention subsequent time, with no concern about over-medicating the patient.

The values and identities of elements of circuit 90 in FIG. 11 are identical to those set forth above in relation to FIG. 9. During active operation, the performance of circuit 90 over a physiologically relevant, or meaningful, range of conductive skin resistances is shown in FIG. 10. The medicament dosage administered by circuit 90 at a skin resistance R_{S}=35 kilo-ohms for various thereby periods conforms to the total medicament dose R_{X} in the table presented in FIG. 10 for circuit 90 of FIG. 9. In combination with those components of circuit 90, the following values and identities of components of timer 100 and switch 104 were found to acceptably disable circuit 90 at the conclusion of typical therapy periods.
C1=single timer UA555 of the type manufactured by Fairchild Semiconductor;
C2=twelve-bit counter 4040 of the type manufactured by Fairchild Semiconductor;
C3=seven-stage ripple cry binary counter 4024 of the type manufactured by Fairchild Semiconductor;
G1=eight-input NAND gate 74LS30 of the type manufactured by Fairchild Semiconductor;
S1=dual-D flip-flop reset switch CD4013 of the type manufactured by Fairchild Semiconductor;
U2=hex inverter amplifier 74LS04 of the type manufactured by Fairchild Semiconductor;
T1=n-p-n transistor 2N3904 of the type manufactured by American Microsemiconductor;
C₁=1 microfarad;
C₂=0.1 microfarads;
R₄=71.5 kilo-ohms;
R₅=1 kilo-ohm; and
V_{T}=5 volts.

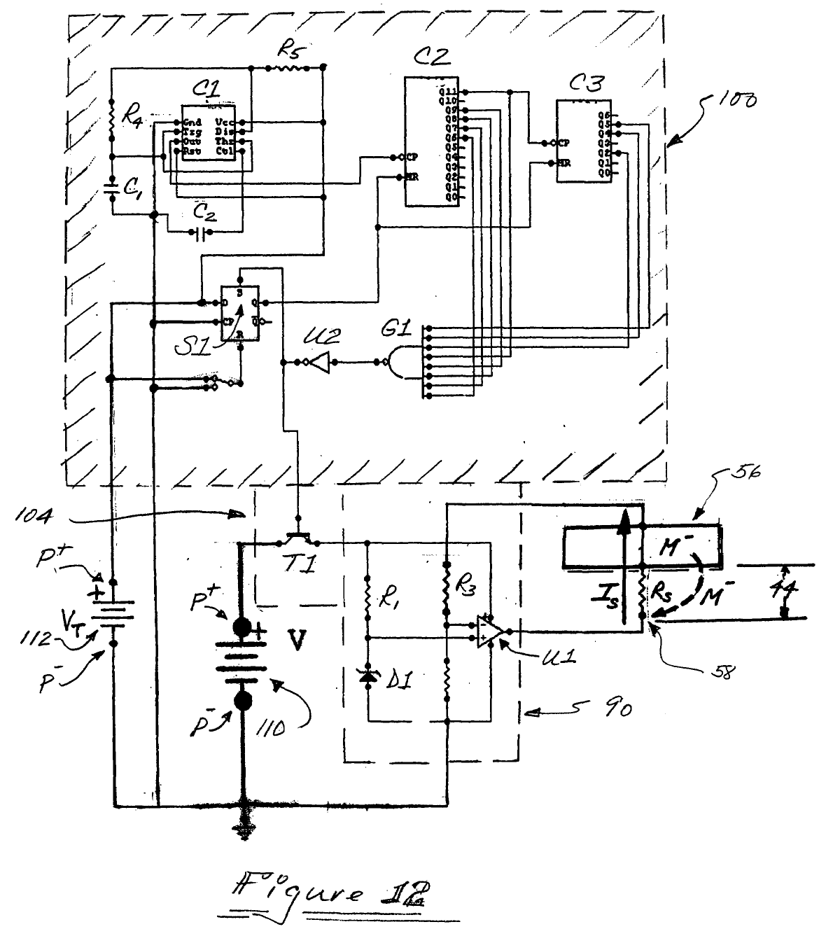

An alternative arrangement for providing power to drive circuit 90 and timer 100 is shown in FIG. 12 in the form of a fifth embodiment of electronics incorporating teachings of the present invention. A primary power source 110 is used to drive circuit 90 through switch 104, and switch 104 is operated by timer 100. In contrast to the arrangement shown in FIG. 11, however, timer 100 is driven exclusively by a supplemental power source 112 that is distinct from primary power source 110 and that is capable at the outset of supplying a timer voltage V_{T} for driving timer 100. The positive pole P⁺ of supplemental power source 112 is coupled directly to timer 100, while the negative poles P⁻ of supplemental power source 112 and primary power source 10 are commonly connected to ground.

The values and identities of the elements of circuit 90 in FIG. 12 are identical to those set forth earlier in relation to FIG. 9, and the values and identities of the elements of timer 100 and switch 104 are identical to those set forth above in relation to FIG. 11. During active operation, the performance of circuit 90 over a physiologically relevant, or meaningful, range of conductive skin resistances is as shown in FIG. 10, and the total dose R_{X} of medicament administered by circuit 90 at a skin resistance R_{S}=35 kilo-ohms for various therapy periods conforms to the medicament doses in the table presented for FIG. 9. Circuit 90 performed as desired using a primary power source 110 having a voltage V=12 volts. Using a supplemental power source 112 having timer voltage V_{T}=3 volts supplied by a single battery, timer 100 and switch 104 were together able to successfully disable circuit 90 at the conclusion of typical therapy periods.

Shown in FIG. 13 is a sixth embodiment of circuitry incorporating teachings of the present invention. There, a circuit 120 is shown that is capable of performing the function of a current means according to teachings of the present invention. Circuit 120 is coupled to the positive pole P⁺ of a primary power source 122, which is capable at the outset of supplying a voltage V for driving circuit 120. Power source 122 causes an electrical current I_{S} to flow through skin 44 of a patient in the direction shown, overcoming in the process electrical skin resistance R_{S} of skin 44. The negative pole P⁻ of power source 62 is coupled, albeit indirectly, to medicament reservoir 56, which is filled, according to the convention set forth above, with molecules of a negative medicament M⁻. As a result, a flow of molecules of medicament M⁻ is induced from reservoir 56, through skin 44, and toward electrical contact 58 in a direction opposite to that of electrical current I_{S}.

Circuit 120 is so configured as to cause electrical current I_{S} to be substantially constant for the full duration of a predetermined therapy period in a range of from 5 to about 8 hours, or more commonly from about 6 hours to about 7 hours. Circuit 120 includes an operational amplifier U1, a zener diode D1, and biasing resistors R₁ and R₂ that are connected as shown. For a skin resistance R_{S}=35 kilo-ohms, the following circuit component values and identities produced a substantially constant electrical current I_{S}=0.230 milliamperes:
U1=operational amplifier LM358 of the type manufactured by Fairchild Semiconductor;
D1=zener diode of the type manufactured by Diodes Incorporated;
R₁=1 kilo-ohm;
R₂=8.66 kilo-ohms; and
V=15 volts.
   In the case of circuit 120, the duration of therapy is controlled by a second embodiment of a timer 124 incorporating teachings of the present invention. Timer 124 is coupled to the positive pole P⁺ of a secondary power source 126, which is capable at the outset of supplying a timer voltage V_{T}=3-5 volts for driving timer 124. Timer 124 includes a single microprocessor M1 that is interconnected with elements of circuit 120 and to secondary power source 126 as shown. Timer 124 initiates the operation of circuit 120 and terminates the operation of circuit 120 after a predetermined therapy period without utilizing any switch intervening between circuit 120 and primary power source 122.

For the circumstances depicted in FIG. 13 and described above, the table that follows sets forth the total dose R_{X} of molecules of medicament M⁻ that would be delivered for several typical therapy periods.

| Therapy Period | Total Dose |
|---|---|
| Duration (hours) | (milliamp-minutes) |
| 5 | 69.0 |
| 6 | 82.8 |
| 7 | 96.6 |
| 8 | 110.4 |

According to yet another aspect of the present invention, a circuit, such as circuit 120, and a timer, such as timer 124, that are included in an active transdermal medicament system are accompanied therein by indicator means carried on the substrate of a patch of that system for signaling when a circuit, such as circuit 120, in operation. As shown by way of example and not limitation in FIG. 13, an indicator means according to teachings of the present invention may take the form of a visual indicator 128, such as a light-emitting diode D2. Optionally, diode D2 may be made by microprocessor M1 to operate intermittently when said circuit 120 is functioning, thereby to conserve the amount of energy required to activate diode D2. As shown in FIG. 13, diode D2 is driven by energy supplied from secondary power source 126 by way of microprocessor M1. Alternatively, a structure performing the function of an indicator means according to teachings of the present invention can be driven by a primary power source, such as primary power source 122, or even provided with a dedicated power source. In combination with the components listed above of circuit 120, timer 124 and indicator 128 were found to acceptably disable circuit 120 at the conclusion of typical therapy periods.

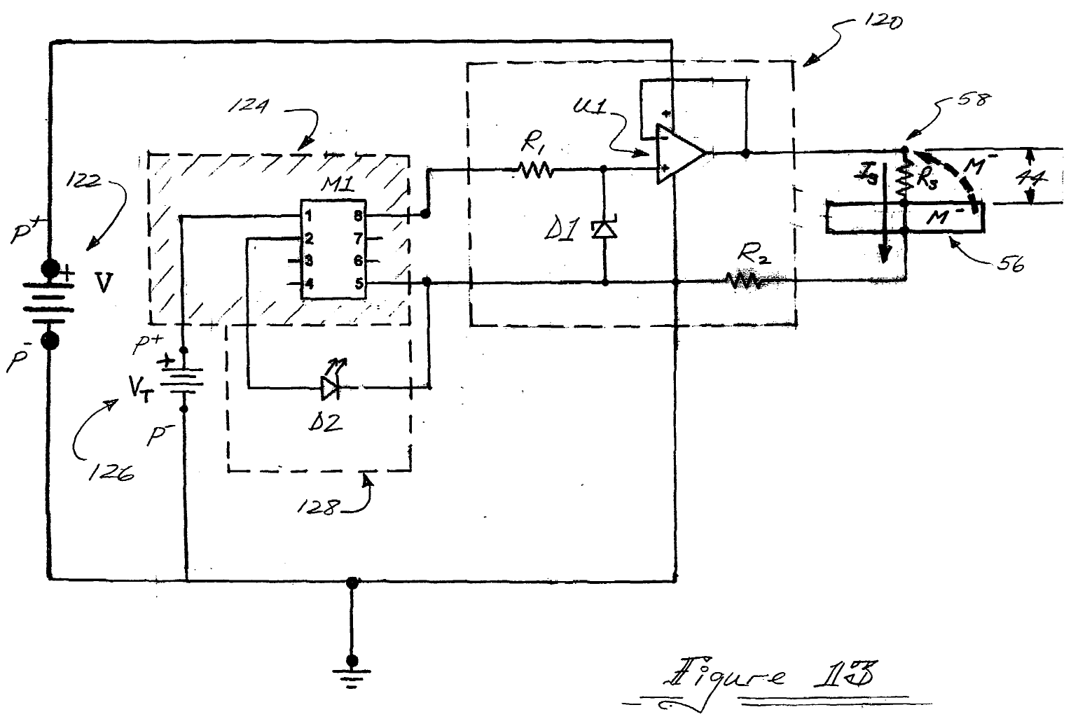

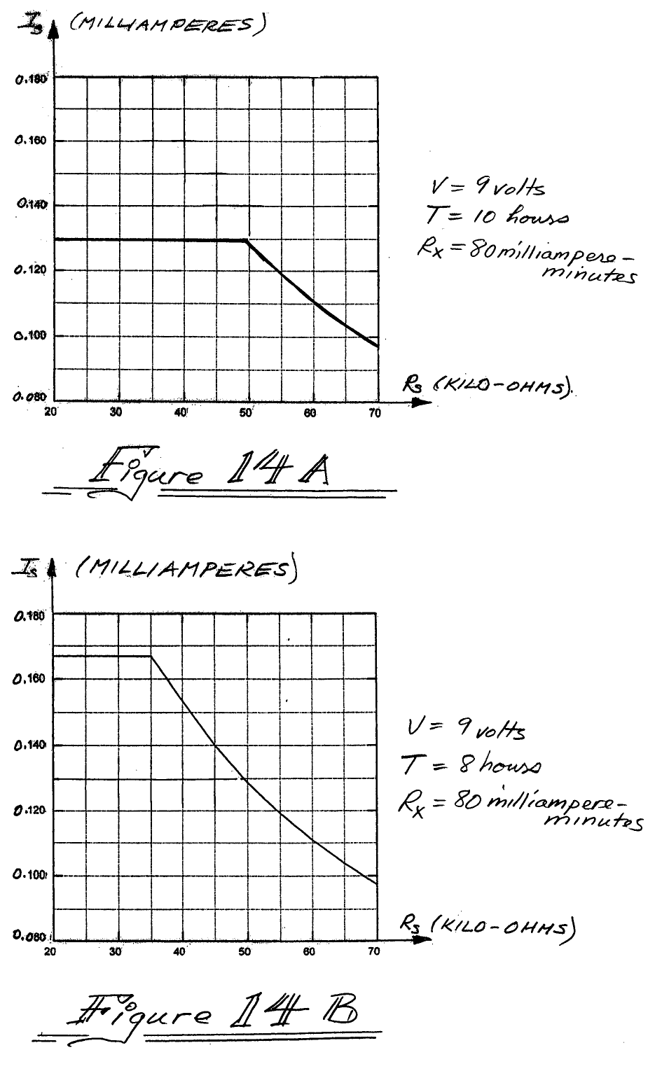

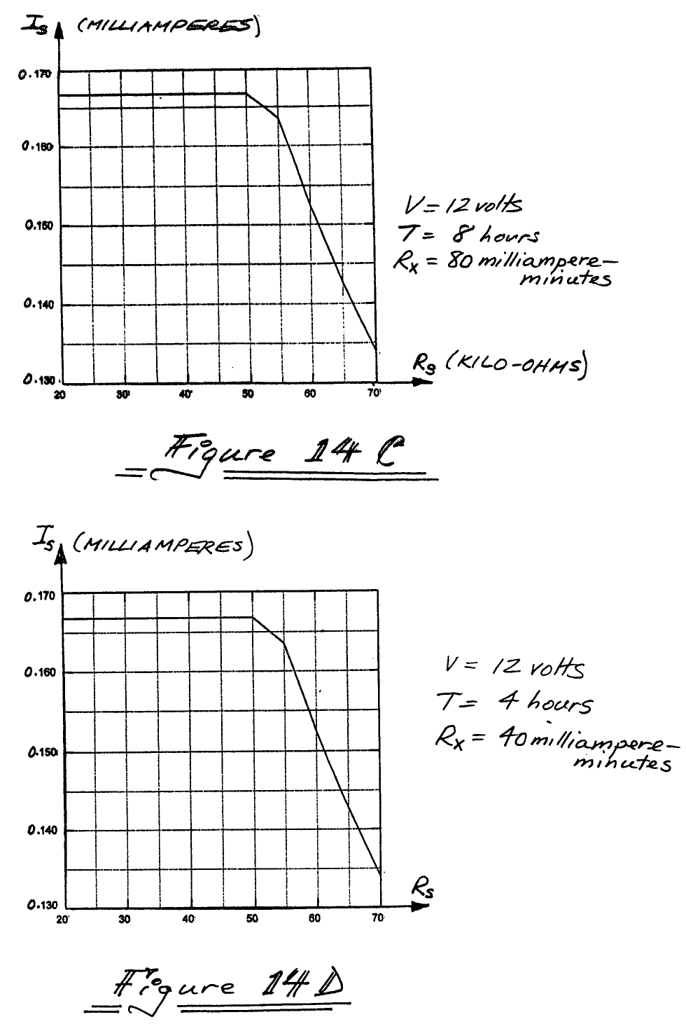

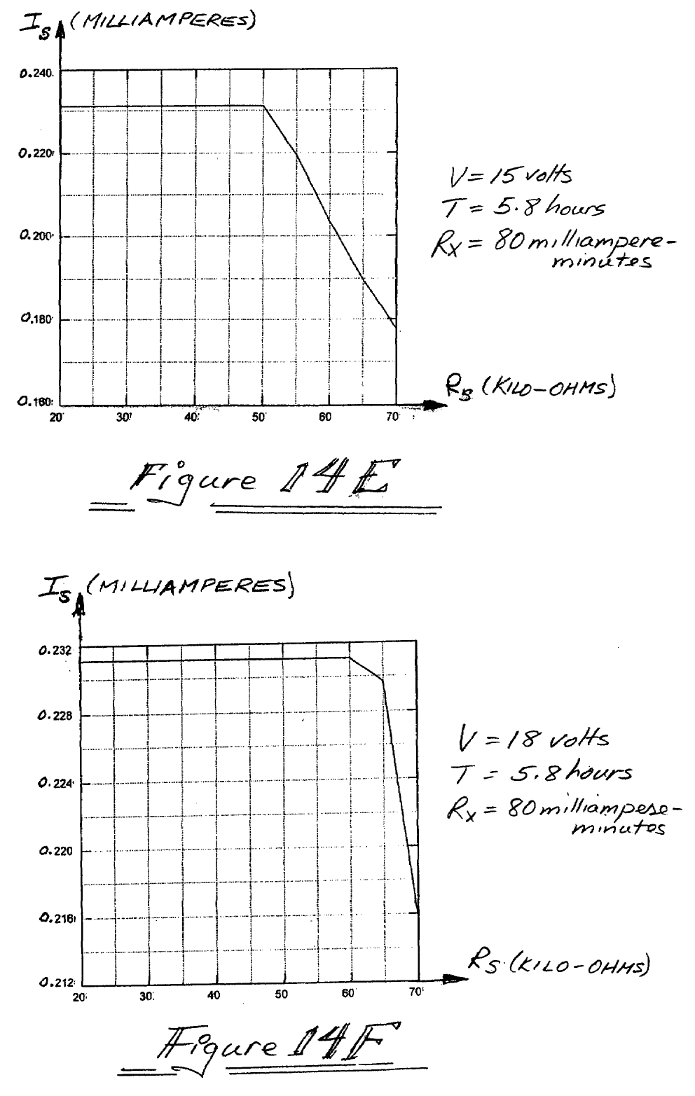

FIGS. 14A-14F are related graphs depicting comparatively the performance of electrical circuit 120 of FIG. 13 across physiologically relevant, or meaningful, ranges of conductive skin resistances R_{S} that are likely to be encountered in patients using an active transdermal patch incorporating teachings of the present invention. These performance graphs were obtained by testing circuit 120 using a known simulation-program-with-integrated-circuit-emphasis, a process that will for convenience and accuracy hereinafter be referred to as “a SPICE test”.

The SPICE tests reflected in FIGS. 14A-14F were conducted using various levels of input voltage V, thereby firstly to ascertain the meaningful range of conductive skin resistance R_{S} over which circuit 120 could supply a substantially unchanged electrical current I_{S} through skin 44 of any given individual patient. Each SPICE test was also used to confirm the ability of a light-weight, self-contained, and non-renewable power source capable at the outset of supplying input voltage V to permit circuit 120 to maintain that electrical current I_{S} for a therapy period T of duration sufficient to deliver various amounts of total medicament dosage R_{X}. Such a power source would be one suitable for carrying on an active transdermal patch incorporating teachings of the present invention.

FIG. 14A is a SPICE test showing the performance of circuit 120 in delivering a total medicament dosage R_{X}=80 milliampere-minutes using a power source capable at the outset of supplying input voltage V=9 volts. A constant electrical current I_{S}=0.130 milliamperes was supplied through skin 44 of a patient for a therapy period T=10 hours. To accomplish this result, it was necessary to employ as biasing resistor R₂=15.38 kilo-ohms. Such conditions were determined to be stably sustainable through a meaningful range of conductive skin resistance R_{S} in skin 44 from about 20 kilo-ohms to about 50 kilo-ohms.

FIG. 14B is a SPICE test showing the performance of circuit 120 in delivering a total medicament dosage R_{X}=80 milliampere-minutes using a power source capable at the outset of supplying input voltage V=9 volts. A constant electrical current I_{S}=0.167 milliamperes was supplied through skin 44 of a patient for a therapy period T=8 hours. To accomplish this result, it was necessary to employ as biasing resistor R₂=11.98 kilo-ohms. Such conditions were determined to be stably sustainable through a meaningful range of conductive skin resistance R_{S} in skin 44 from about 20 kilo-ohms to about 35 kilo-ohms.

FIG. 14C is a SPICE test showing the performance of circuit 120 in delivering a total medicament dosage R_{X}=80 milliampere-minutes using a power source capable at the outset of supplying input voltage V=12 volts. A constant electrical current I_{S}=0.167 milliamperes was supplied through skin 44 of a patient for a therapy period T=8 hours. To accomplish this result, it was necessary to employ as biasing resistor R₂=11.98 kilo-ohms. Such conditions were determined to be stably sustainable through a meaningful range of conductive skin resistance R_{S} in skin 44 from about 20 kilo-ohms to about 50 kilo-ohms.

FIG. 14D is a SPICE test showing the performance of circuit 120 in delivering a total medicament dosage R_{X}=40 milliampere-minutes using a power source capable at the outset of supplying input voltage V=12 volts. A constant electrical current I_{S}=0.167 milliamperes was supplied through skin 44 of a patient for a therapy period T=4 hours. To accomplish this result, it was necessary to employ as biasing resistor R₂=11.98 kilo-ohms. Such conditions were determined to be stably sustainable through a meaningful range of conductive skin resistance R_{S} in skin 44 from about 20 kilo-ohms to about 50 kilo-ohms.

FIG. 14E is a SPICE test showing the performance of circuit 120 in delivering a total medicament dosage R_{X}=80 milliampere-minutes using a power source capable at the outset of supplying input voltage V=15 volts. A constant electrical current I_{S}=0.232 milliamperes was supplied through skin 44 of a patient for a therapy period T=5.8 hours. To accomplish this result, it was necessary to employ as biasing resistor R₂=8.62 kilo-ohms. Such conditions were determined to be stably sustainable through a meaningful range of conductive skin resistance R_{S} in skin 44 from about 20 kilo-ohms to about 50 kilo-ohms.

FIG. 14F is a SPICE test showing the performance of circuit 120 in delivering a total medicament dosage R_{X}=80 milliampere-minutes using a power source capable at the outset of supplying input voltage V=18 volts. A constant electrical current I_{S}=0.230 milliamperes was supplied through skin 44 of a patient for a therapy period T=5.8 hours. To accomplish this result, it was necessary to employ as biasing resistor R₂=8.69 kilo-ohms. Such conditions were determined to be stably sustainable through a meaningful range of conductive skin resistance R_{S} in skin 44 from about 20 kilo-ohms to about 60 kilo-ohms.

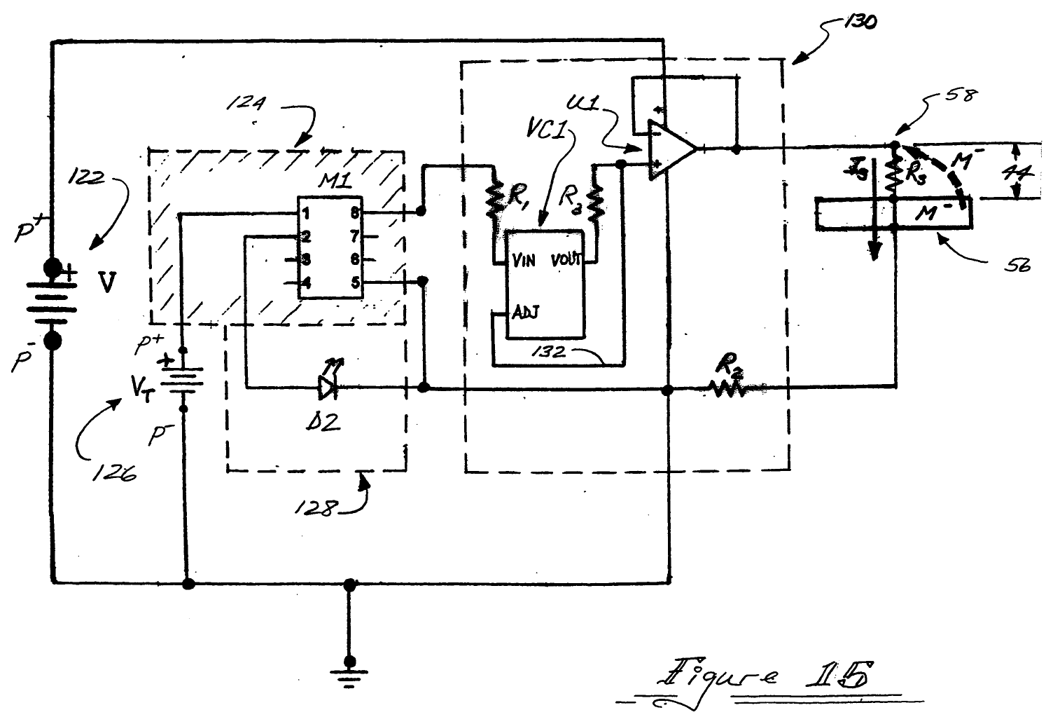

By way of example and not limitation, shown in FIG. 15 is a schematic diagram of a seventh embodiment of electronics incorporating teachings of the present invention. Shown is a circuit 130 of capable of performing the function of a current means according to teachings of the present invention. Circuit 130 is coupled to the positive pole P⁺ of power source 62, which is capable at the outset of supplying a voltage V for driving circuit 130. Power source 62 causes an electrical current I_{S} to flow through skin 44 of a patient in the direction shown, overcoming in the process electrical skin resistance R_{S} of skin 44. The negative pole P⁻ of power source 62 is coupled, albeit indirectly, to medicament reservoir 56, which is filled, according to the convention set forth above, with molecules of a negative medicament M⁻. As a result, a flow of molecules of medicament M⁻ is induced from reservoir 56, through skin 44, and toward electrical contact 58 in a direction opposite to that of electrical current I_{S}.

Circuit 130 is so configured as to cause electrical current I_{S} to be substantially constant for the full duration of a predetermined therapy period in a range of from 5 to about 8 hours, or more commonly from about 6 hours to about 7 hours. Circuit 130 differs from circuit 120 of FIG. 13 by including a voltage regulator VC1 and a feedback resistor R₃ in place of zener diode D1 in circuit 120. Thus, circuit 130 includes a second embodiment of a voltage reference means according to teachings of the present invention for providing a substantially invariant voltage to the positive input terminal VIN of operational amplifier U1.

According to another aspect of the present invention, voltage regulator VC1 is provided with regulator output voltage stabilization means for maintaining the delivery of a substantially constant voltage by voltage regulator VC1 to the positive input terminal of operational amplifier U1. As shown by way of example and not limitation in FIG. 15, feedback resistor R₃ is coupled between the output terminal VOUT of voltage regulator VC1 and the positive input terminal of operational amplifier U1. A feedback loop is provided between the positive input terminal of operational amplifier U1 and an auxiliary input terminal AUX of voltage regulator VC1. The feedback loop takes the form of feedback signal line 132 that is coupled between the positive input terminal of operational amplifier U1 and auxiliary input terminal ADJ of voltage regulator VC1.

Other than for voltage regulator VC1 and feedback resistor R₃, the values and identities of the components of circuit 130 are identical to those set forth earlier relative to circuit 120 in FIG. 13. For a skin resistance R_{S}=35 kilo-ohms, the following circuit component values and identities produced a substantially constant electrical current I_{S}=0.230 milliamperes:
VC1=voltage regulator LM317 of the type manufactured by Fairchild Semiconductor;
R₃=6.98 kilo-ohm; and
V=15 volts.

In the case of circuit 130, the duration of therapy is controlled by timer 124 as described above relative to FIG. 13. During any therapy period, visual indicator 128, which also described earlier relative to FIG. 13, gives notice to medical personnel that circuit 130 is functioning. For the circumstances depicted in FIG. 15 and described above, the table that follows sets forth the total dose R_{X} of molecules of medicament M⁻ that would be delivered for several typical therapy periods.

| Therapy Period | Total Dose |
|---|---|
| Duration (hours) | (milliampere-minutes) |
| 5 | 69.0 |
| 6 | 82.8 |
| 7 | 96.6 |
| 8 | 110.4 |

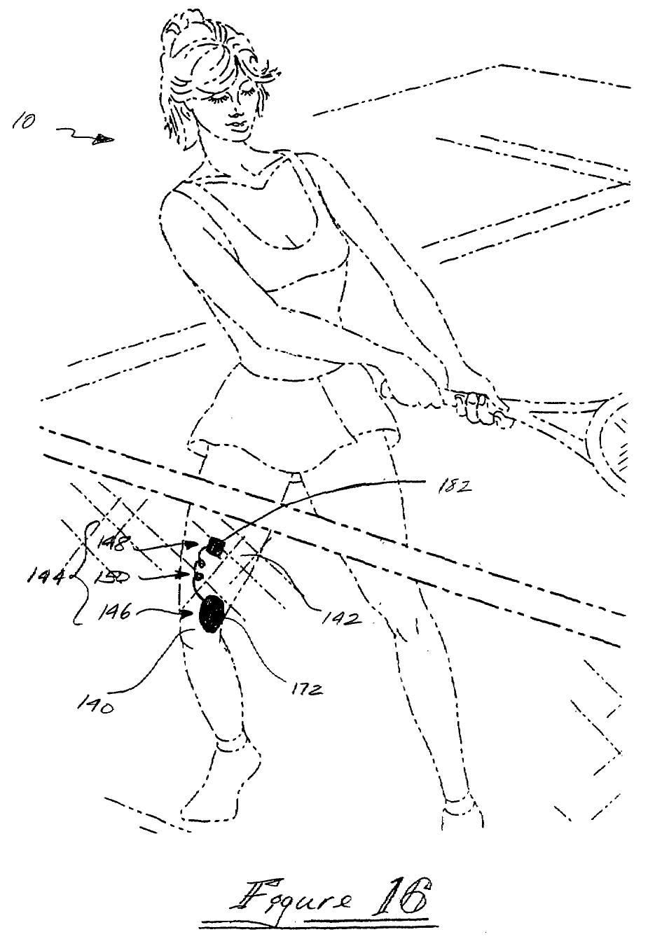

FIG. 16 shows patient 10 again requiring the localized administration of a medicament, but in this instance to knee 130 and thigh 132 thereof. For that purpose, patient 10 is wearing on knee 130 and thigh 132 thereof elements of a second embodiment of an active iontophoretic delivery system 144 that incorporates teachings of the present invention. While so doing, patient 10 is nonetheless able to engage in vigorous physical activity, because delivery system 144 is entirely self-contained, and not supplied with power from any immobile or cumbersome power source. Delivery system 144 includes an active transdermal medicament patch 146, a distinct electrical contact 148, and a contact tether 150 that structurally interconnects electrical contact 148 with patch 146.

Patch 146 is removable adhered to the skin of knee 130 of patient 10 at the location at which the need for the administration of medicament is most acute, while electrical contact 148 is removable adhered to the skin of thigh 132 of patient 10 at a location remote from patch 146. These elements of delivery system 144 are worn interconnected by tether 150 for the duration of a predetermined therapy period. The length of the therapy period during which patch 146 and electrical contact 148 must be worn is determined by the rate at which patch 146 delivers medicament through the skin of patient 10 and the total dose of medicament that is to be administered.

FIGS. 17-20 taken together afford an overview of the structure of the elements of delivery system 144.

FIG. 17 reveals that patch 146 of delivery system 144 includes a flexible, planar biocompatible substrate 152 having a therapeutic face 154 on one side thereof that is intended to be disposed in contact with the skin of patient 10. Therapeutic face 154 is coated with a biocompatible adhesive and is for that reason removable securable to the skin of patient 10. Prior to the actual use of delivery system 144, the adhesive on therapeutic face 154 of patch 146 is shielded by a removable first release liner 156, which is shown in FIG. 17 in the process of being peeled away from therapeutic face 154. Formed centrally through release liner 156 is a medicament aperture 158. The function of medicament aperture 158 will be explained in due course.

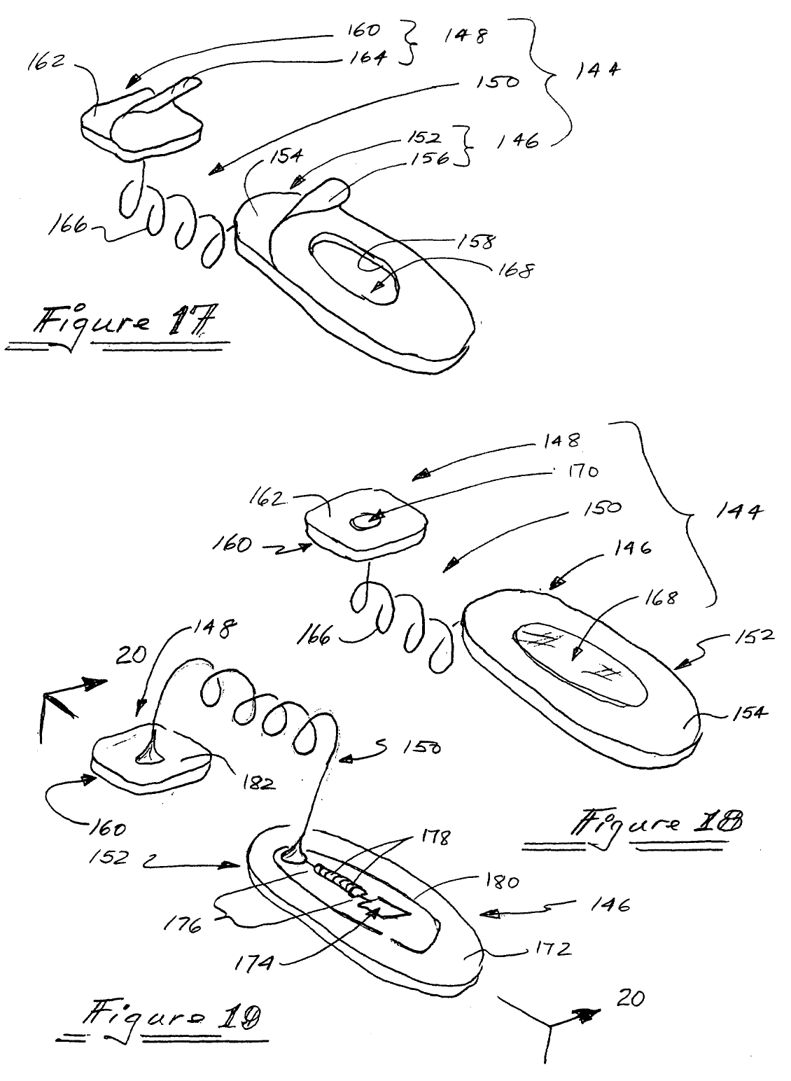

Similarly, as shown in FIG. 17, electrical contact 148 of delivery system 144 includes a flexible, planar biocompatible substrate 160 having a therapeutic face 162 on one side thereof that is intended to be disposed in contact with the skin of patient 10. Therapeutic face 162 is coated with a biocompatible adhesive and is for that reason removable securable to the skin of patient 10. Prior to the actual use of delivery system 144, the adhesive on therapeutic face 162 of electrical contact 148 is shielded by a removable second release liner 164, which is also shown in FIG. 18 in the process of being peeled away from therapeutic face 162.

Finally, in FIG. 17 tether 150 is shown extensibly attaching patch 146 to electrical contact 148, thereby to permit electrical contact 148 to be positioned on the skin of patient 10 at any reasonable distance from patch 146 and in any direction there patch 146 therefrom. While tether 150 maintains the physical interconnectedness between patch 146 and electrical contact 148, is also the function of tether 150 to electrically couple electrical components of delivery system 144 carried on patch 146 with electrical components of delivery system 144 carried on electrical contact 148. Accordingly, as seen in FIG. 17, an embodiment of tether 150 capable of performing both of these functions is shown to be an insulated electrical conductor 166 of length sufficient to enable electrical contact 148 to be disposed against the skin of patient 10 at a locating remote from patch 146.

In alternative embodiments of delivery system 144, a tether, such as tether 150, incorporating teachings of the present invention might comprise a materially continuous elongation of substrate 152 of patch 146 or a materially continuous elongation of substrate 160 of electrical contact 148. That material elongation would then need to carry thereon or have embedded therein an electrically conductive pathway, such as conductor 166, that serves to electrically couple the electrical components of delivery system 144 carried on patch 146 with the electrical components of delivery system 144 carried on electrical contact 148. It is even conceivable that a tether according to teachings of the present invention could be but a portion of a single, materially uniform structure that encompasses both substrate 152 of patch 146 and substrate 160 of electrical contact 148 and that carries or has embedded therein an electrically conductive pathway like conductor 166.

FIG. 18 depicts features of delivery system 144 that are revealed upon the removal of first release liner 156 from therapeutic face 154 of first substrate 152 and the removal of second release liner 164 from therapeutic face 162 of second substrate 160.

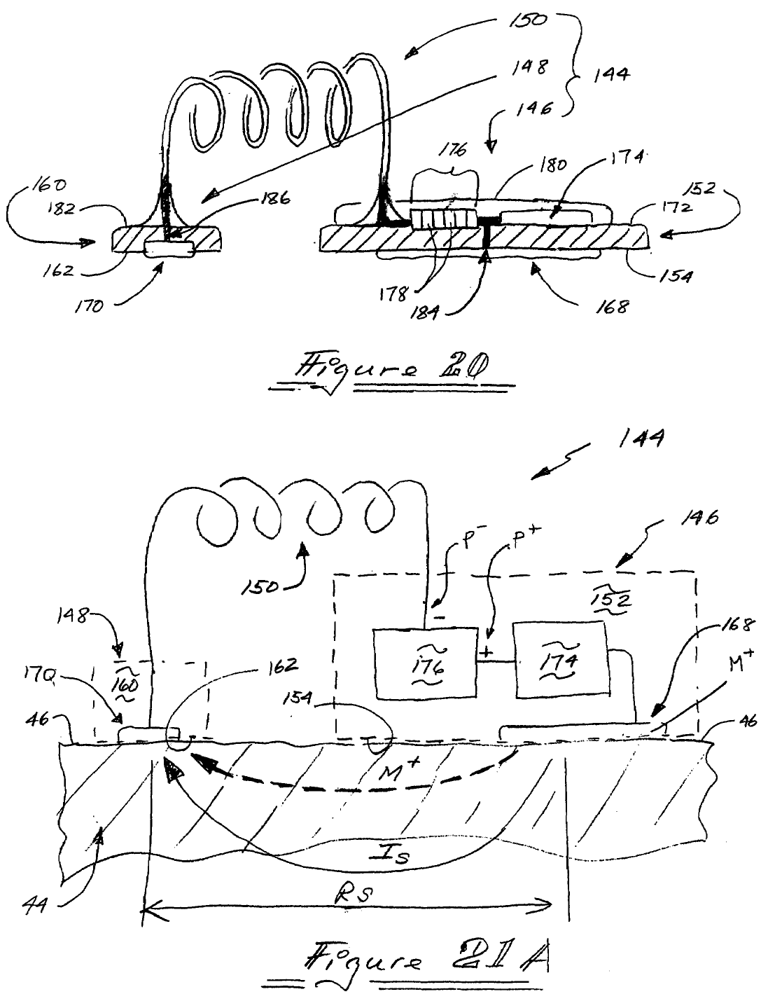

In FIG. 19 it can bee seen that patch 146 includes a medicament reservoir 168 that is positioned on therapeutic face 154 of first substrate 152 interior of the periphery of therapeutic face 154. Reservoir 168 is intended to electrically conductively engage the skin of patient 10, when therapeutic face 154 of first substrate 152 is disposed against the skin of patient 10. Reservoir 168 can take the form of a gel suspension of medicament or a pad of gauze or cotton saturated with fluid containing medicament. It is the purpose of medicament aperture 158 in first release liner 156 to permit the disposition of medicament in reservoir 168 prior to the removal of first release liner 156 from therapeutic face 20 of substrate 18.

FIG. 18 also reveals that an electrical contact pad 170 is positioned on therapeutic face 162 of second substrate 160 of electrical contact 148. Contact pad 170 is separated from reservoir 168 to any degree enabled by tether 150. Contact pad 170 is thus electrically isolated from reservoir 168. Contact pad 170 is also capable of electrically conductively engaging the skin of patient 10 when therapeutic face 162 of second substrate 160 is disposed against the skin. Accordingly, when as shown in FIG. 17, patch 146 and electrical contact 148 are adhered to the skin of patient 10, contact pad 170 engages the skin of patient 10 at a location that is remote from reservoir 168.

FIG. 19 shows the upper face 172 of first substrate 152, which is the face of first substrate 152 on the opposite side of first substrate 152 from therapeutic face 154 shown in FIGS. 17 and 18. Upper face 172 is thus the face of first substrate 152 that is visible when worn by patient 10 in FIG. 17. Upper face 172 of first substrate 152 carries electronic circuitry 154 and a corresponding power source 176, which is shown by way of example as being a roll of series-connected miniature batteries 178, each of about 3 volts potential. Power source 176 thus supplies non-alternating current to electronic circuitry 174. Electronic circuitry 174 and power source 176 are shown as being encased on upper face 172 of first substrate 152 by a transparent protective cover 180, but either or both of power source 176 and electronic circuitry 174 could with equal functional adequacy be partially or wholly imbedded in first substrate 152, or even carried on therapeutic face 154 thereof.

Upper face 172 of first substrate 152 is connected by tether 150 to the upper face 182 of second substrate 160, which is the face of second substrate 160 on the opposite side of second substrate 160 from therapeutic face 162 shown in FIGS. 18 and 19. Upper face 182 is thus the face of second substrate 160 that is visible when worn by patient 10 in FIG. 16.

In the embodiment of a medicament delivery system depicted in FIGS. 16-19, electronic circuitry 154 and power source 176 are both located on the same substrate of delivery system 144 that carries reservoir 168. Nonetheless, such a relationship need not be maintained among elements in a medicament delivery system embodying teachings of the present invention. One or both of electronic circuitry 154 and power source 176 could be located apart from reservoir 168 with contact pad 170 on second substrate 160 and then electrically interconnected with other electrical elements of delivery system 144 on first substrate 152 by tether 150.

FIG. 20 is an elevation cross section view of patch 146 and electrical contact 148 of delivery system 144 taken along section line 20-20 in FIG. 190. As a result, FIG. 20 depicts in edge view both sides of first substrate 152 and second substrate 160, as well as the interaction through each of the features of delivery system 144 shown in FIGS. 16-19. Reservoir 168 is shown as being carried on therapeutic face 154 of first substrate 152, while electronic circuitry 174 and power source 176 encased in cover 180 are shown carried on upper face 172. One of electronic circuitry 174 and power source 176 is electrically interconnected by way of a first via 184 through first substrate 152 to reservoir 168. If either or both of power source 176 and electronic circuitry 174 is partially or wholly imbedded in first substrate 152 or carried on therapeutic face 154, the need for first vial 84 may be obviated.

Similarly, contact pad 170 is shown as being carried on therapeutic face 162 of second substrate 160 and electrically interconnected by way of a second via 186 through second substrate 160 to upper face 182 and one end of tether 150. The opposite end of tether 150 is connected to upper face 172 of first substrate 152 and the electronic components of delivery system 144 carried thereon. In any case, reservoir 168 and contact pad 170 are electrically isolated from each other.

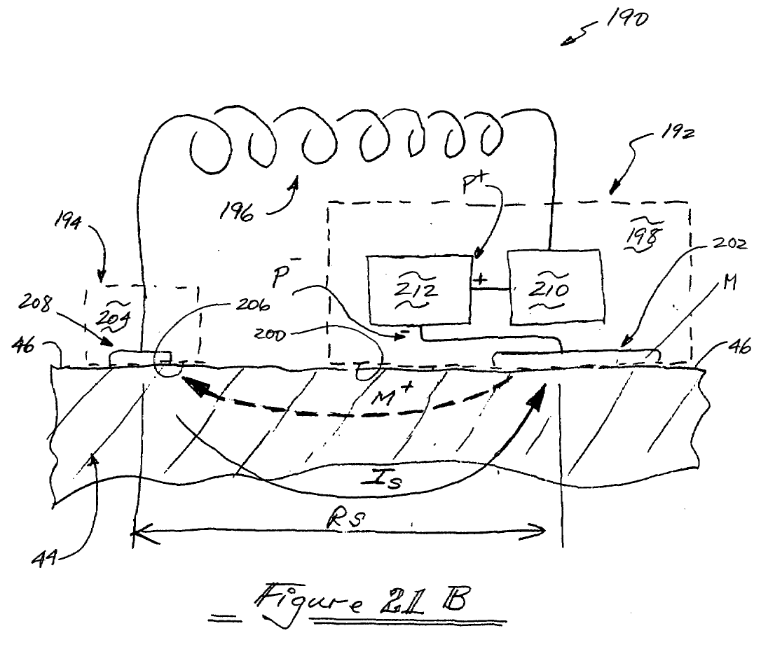

FIGS. 21A and 21B are related diagrams that compare the movement of medicaments of differing polarities through the skin of a wearer of delivery system 144 and the altered electrical interconnections required among element of delivery system 144 to produce those movements.

FIG. 21A illustrates the movement molecules of a positive medicament M⁺ that when dissolved exhibits a net positive polarity. Therapeutic face 154 of first substrate 152 of patch 146 is shown as being disposed against surface 46 of skin 44. Therapeutic face 162 of second substrate 160 of electrical contact 148 is also shown as being disposed against surface 46 of skin 44. Thus reservoir 168 and contact pad 170 each electrically conductively engage surface 46 of skin 44, but at locations that are separated from each other. Aside from the conductivity of skin 44, these locations are electrically isolated from each other.

The positive pole P⁺ of power source 176 is coupled through electronic circuitry 174 to reservoir 168. The negative pole P⁻ of power source 34 is coupled by way of tether 150 directly to contact pad 170. The electromotive differential thusly applied to skin 44 between reservoir 168 and contact pad 170 induces molecules of positive medicament M⁺ to move as positive ions out of reservoir 168 toward skin 44, across the unbroken surface 46 of skin 44, and through skin 44 in the direction of contact pad 170. This movement is indicated in FIG. 21A by a dashed arrow labeled M⁺. A skin current I_{S} is schematically indicated by a solid arrow to flow through skin 44 from reservoir 168 that is associated with positive pole P⁺ of power source 176 to contact pad 170 that is associated with negative pole P⁻ of power source 176. The presence of electrical resistance in skin 44 is indicated schematically in FIG. 21A as skin resistance R_{S}.

In FIG. 21B, transcutaneous administration is intended of molecules of a negative medicament M⁻ that in solution exhibits a net negative polarity. Under such conditions the electrical components of an active medicament delivery system incorporating teachings of the present invention must be altered from those shown in FIG. 21A. Accordingly, in FIG. 21B a second embodiment of an active medicament delivery system 190 incorporating teachings of the present invention is shown to include an active transdermal medicament patch 192, a distinct electrical contact 194, and a contact tether 196 that structurally and electrically interconnects electrical contact 194 with patch 192. Patch 192 includes a first substrate 198 having a therapeutic face 200 that carries a medicament reservoir 202. Electrical contract 194 includes a second substrate 204 having a therapeutic face 206 that carries a contact pad 208. Electronic circuitry 210 and a power source 212 are included in patch 192, and reservoir 202 is filled with molecules of a negative medicament M⁻.

Therapeutic face 200 of first substrate 198 of patch 192 is shown as being disposed against surface 46 of skin 44, and therapeutic face 206 of second substrate 204 of patch 192 is also shown as being disposed against surface 46 of skin 44. Reservoir 202 and contact pad 208 each electrically conductively engage surface 46 of skin 44, but at separated locations. Aside from the conductivity of skin 44, these locations are electrically isolated from each other. Positive pole P⁺ of power source 212 is coupled through electronic circuitry 210 and tether 196 to contact pad 208. Correspondingly, negative pole P⁻ of power source 212 is coupled directly to reservoir 202. The electromotive differential thusly applied to skin 44 between contact pad 208 and reservoir 202 induces molecules of negative medicament M⁻ to move as negative ions out of reservoir 202 toward skin 44, across the unbroken surface 46 of skin 44, and through skin 44 in the direction of contact pad 208. This movement is indicated in FIG. 21B by a dashed arrow labeled M⁻. A skin current I_{S} is schematically indicated by a solid arrow to flow through skin 44 toward reservoir 202, which is associated with negative pole P⁻ of power source 212, from contact pad 208, which is associated through electronic circuitry 210 and by way of tether 196 with positive pole P⁺ of power source 212. The presence of electrical resistance in skin 44 is indicated schematically as skin resistance R_{S}.

According to one aspect of the present invention, an active transdermal medicament delivery system, such as delivery system 144 in FIGS. 17-21A or delivery system 190 in FIG. 21B, includes current means carried on one of the substrates of the system that is driven by a power source that is also carried on one of those substrates. The current means is for causing a substantially constant current to flow through the medicament reservoir of the delivery system and the skin of a wearer of the delivery system. In this manner, the total dose of medicament delivered by an active transdermal medicament delivery system incorporating teachings of the present invention is reliable determinable by reference to the total time during which the delivery system is employed for therapy.

By way of example and not limitation, shown in FIG. 7 and discussed above is a first embodiment of a circuit 70 capable of performing the function of a current means according to teachings of the present invention. Shown in FIG. 8 and discussed above is a second embodiment of a circuit 80 also capable of performing the function of a current means according to teachings of the present invention. Shown in FIG. 9 and discussed above is a third embodiment of a circuit 90 also capable of performing the function of a current means according to teachings of the present invention. Shown in FIG. 13 and discussed above is a fourth embodiment of a circuit 120 capable of performing the function of a current means according to teachings of the present invention. Shown in FIG. 15 and discussed above is a fourth embodiment of a circuit 130 capable of performing the function of a current means according to teachings of the present invention.

According to yet another aspect of the present invention, an electric circuit, such as circuits 70, 80, 90, 120, or 130 is accompanied in an active iontophoresis medicament delivery system by control means carried on one of the substrates of that system. The control means is for disabling the electric circuit following the operation of that circuit for a predetermined therapy period. Thus, the inclusion in an active iontophoresis medicament delivery system of a control means according to teachings of the present invention enables the automatic control of the duration of therapy, regardless of the amount of time that the system is in contact with the skin of a patient. This eliminates any need to note with specificity the time at which the system is first disposed against the surface of the skin of the patient, or to remove the system promptly at the end of the chosen therapy period.

By way of illustration and not limitation, as shown in FIGS. 11 and 12 and discussed above, a circuit according to teachings of the present invention is provided with a timer 100 that performs the functions of a control means according to teachings of the present invention. Shown in FIG. 13 and discussed above, a circuit according to teachings of the present invention is provided with a second embodiment of a timer 124 that performs the functions of a control means according to teachings of the present invention.

Finally, the present invention also includes the methods of manufacture necessary to provide and of the inventive embodiments described above, as well as methods associated with the effective therapeutic use of any of those inventive embodiments.

The invention may be embodied in other specific forms without departing from its spirit or essential characteristics. The described embodiments are to be considered in all respects only as illustrative and not restrictive. The scope of the invention is, therefore, to be defined by the appended claims, rather than by the foregoing description. All variations from the literal recitations of the claims that are, nonetheless, within the range of equivalency correctly attributable to the literal recitations are, however, to be considered to be within the scope of those claims.

1. An active transdermal medicament patch comprising:
(a) a flexible, planar biocompatible substrate having a therapeutic face on one side thereof configured for disposition against the skin of a patient;
(b) a medicament reservoir positioned on said therapeutic face of said substrate, said reservoir electrically conductively engaging the skin of a patient when said therapeutic face of said substrate is disposed there against;
(c) a power source carried on said substrate;
(d) current means non-removably carried on said substrate and driven by said power source for causing a substantially constant current through said reservoir and the skin of a patient during the course of a predetermined therapy period; and
(e) control means non-removably carried on said substrate for disabling said current means following functioning of said current means for said therapy period.

2. A medicament patch as recited in claim 1, wherein said therapeutic face of said substrate is configured for removable securement to the skin of a patient.

3. A medicament patch as recited in claim 2, wherein said therapeutic face of said substrate is coated with a biocompatible adhesive.

4. A medicament patch as recited in claim 3, further comprising a release liner covering said adhesive.

5. A medicament patch as recited in claim 1, wherein said medicament reservoir is positioned interior of the periphery of said therapeutic face of said substrate.

6. A medicament patch as recited in claim 1, further comprising an electrical contact positioned on said therapeutic face of said substrate electrically isolated from said reservoir, said contact being capable of electrically conductively engaging the skin of a patient when said therapeutic face of said substrate is disposed there against.

7. A medicament patch as recited in claim 6, wherein said power source is electrically coupled between said current means and said electrical contact.

8. A medicament patch as recited in claim 6, wherein said power source is electrically coupled between said current means and said medicament reservoir.

9. A medicament patch as recited in claim 1, wherein said power source produces non-alternating power.

10. A medicament patch as recited in claim 9, wherein said power source comprises a plurality of series-connected batteries.

11. A medicament patch as recited in claim 9, wherein said power source comprises a direct current alternator.

12. A medicament patch as recited in claim 1, wherein said control means is driven by said power source.

13. A medicament patch as recited in claim 1, further comprising an auxiliary power source carried on said substrate, said control means being driven by said auxiliary power source.

14. A medicament patch as recited in claim 1, wherein said control means comprises a timer.

15. A medicament patch as recited in claim 1, further comprising indicator means non-removably carried on said substrate for signaling when said current means is functioning.

16. A medicament patch as recited in claim 15, wherein said indicator means comprises a visual indicator.

17. A medicament patch as recited in claim 16, wherein said visual indicator is a light-emitting diode.

18. A medicament patch as recited in claim 17, wherein said light-emitting diode operates intermittently during said therapy period.

19-27. (canceled)

28. An active transdermal medicament patch as recited in claim 1, wherein said current means non-removably carried on said substrate and driven by said power source for delivering a substantially constant current through said reservoir and the skin of a patient during the course of a predetermined therapy period does so for a patient having a current flow skin resistance within a meaningful range of current flow skin resistances.

29. A medicament patch as recited in claim 28, wherein said meaningful range of current flow skin resistances comprises current flow skin resistances in a range of from about 20 kilo-ohms to about 70 kilo-ohms.

30. A medicament patch as recited in claim 29, wherein said meaningful range of current flow skin resistances comprises current flow skin resistances in a range of from about 25 kilo-ohms to about 50 kilo-ohms.

31. A medicament patch as recited in claim 30, wherein said meaningful range of current flow skin resistances comprises current flow skin resistances in a range of from about 30 kilo-ohms to about 40 kilo-ohms.

32. A medicament patch as recited in claim 30, wherein variation in said substantially constant current is limited by said current means to plus or minus 5% of a target therapeutic current flow.

33. A medicament patch as recited in claim 32, wherein said target therapeutic current flow is equal to about 230 milliamperes.

34. A medicament patch as recited in claim 32, wherein variation in said substantially constant current is limited by said current means to plus or minus 2% of a target therapeutic current flow.

35. A medicament patch as recited in claim 34, wherein said target therapeutic current flow is equal to about 230 milliamperes.

36. A medicament patch as recited in claim 28, wherein at the commencement of said therapy period, the output of said power source is direct current at an electromotive force in a range of from about 9 volts to about 18 volts.

37. A medicament patch as recited in claim 36, wherein said power source comprises a plurality of series-connected batteries.

38. A medicament patch as recited in claim 36, wherein at the commencement of said therapy period, the output of said power source is direct current at an electromotive force of about 12 volts

39. A medicament patch as recited in claim 38, wherein said power source comprises a plurality of series-connected batteries.

40. A medicament patch as recited in claim 28, wherein the duration of said therapy period is in a range of from about 5 hours to about 8 hours.

41. A medicament patch as recited in claim 40, wherein the duration of said therapy period is about 7 hours.

42. A medicament patch as recited in claim 28, further comprising indicator means non-removably carried on said substrate for signaling when said current means is functioning.

43. A medicament patch as recited in claim 28, further comprising a supplementary power source carried on said substrate dedicated to delivering power to said control means.

44. A medicament patch as recited in claim 43, further comprising indicator means non-removably carried on said substrate for signaling when said current means is functioning, said indicator means being driven by said supplementary power source.

45. A medicament patch as recited in claim 28, wherein said power source, said reservoir, and said current means are so interconnected as to enable said medicament patch to deliver into the skin of a patient a positive polarity medicament disposed in said reservoir.

46. A medicament patch as recited in claim 28, wherein said power source, said reservoir, and said current means are so interconnected as to enable said medicament patch to deliver into the skin of a patient a negative polarity medicament disposed in said reservoir.

47. An active transdermal medicament patch as recited in claim 1, wherein said current means comprises an electric circuit non-removably carried on said substrate, said electric circuit being driven by said power source and being series-connected with said reservoir and the skin of a patient, said electrical circuit thereby delivering through said reservoir and the skin of a patient a substantially constant current of about 230 plus or minus 10 milliamps during the course of a predetermined therapy period for a patient having a current flow skin resistance within a meaningful range of current flow skin resistances from about 20 kilo-ohms to about 70 kilo-ohms.

48. A medicament patch as recited in claim 47, further comprising control means non-removably carried on said substrate for disabling said electric circuit following functioning of said electric circuit for said therapy period.

49. A medicament patch as recited in claim 48, wherein said control means comprises a timer.

50. A medicament patch as recited in claim 49, wherein said timer comprises a flip-flop down-counter.

51. A medicament patch as recited in claim 49, wherein said timer is coupled to the gate of a transistor source-to-drain series-connected between said electric circuit and said power source.

52. A medicament patch as recited in claim 51, wherein said timer is driven by a tap from said power source.

53. A medicament patch as recited in claim 51, wherein said timer is driven by a supplemental power source.

54. A medicament patch as recited in claim 52, wherein said supplemental power source comprises a three-volt battery.

55. A medicament patch as recited in claim 47, wherein at the commencement of said therapy period, said power source delivers direct current at an electromotive force in a range of from about 9 volts to about 18 volts.

56. A medicament patch as recited in claim 47, wherein said electric circuit is coupled through said reservoir to the skin of a patient.

57. A medicament patch as recited in claim 47, wherein said electric circuit is coupled through the skin of a patient to said reservoir.

58. A medicament patch as recited in claim 57, wherein said electric circuit comprises a field effect transistor source-to-drain series-connected between said power source and the skin of a patient.

59. A medicament patch as recited in claim 58, wherein the gate of said field effect transistor is coupled through a tuning resistor to said power source.

60. (canceled)

61. A medicament patch as recited in claim 47, wherein said electric circuit comprises:
(a) an operational amplifier; and
(b) amplifier output current stabilization means for maintaining the delivery of said substantially constant current by said operational amplifier through said reservoir and the skin of a patient.

62-69. (canceled)

70. An active transdermal medicament patch comprising:
(a) a flexible, planar biocompatible substrate having a therapeutic face on one side thereof configured for disposition against the skin of a patient:
(b) a medicament reservoir positioned on said therapeutic face of said substrate said reservoir electrically conductively enaaging the skin of a patient when said therapeutic face of said substrate is disposed there against;
(c) a power source carried on said substrate; and
(d) an electric circuit non-removably carried on said substrate, said electric circuit being driven by said power source and being series-connected with said reservoir and the skin of a patient when said therapeutic face of said substrate is disposed there against, said electrical circuit thereby delivering a substantially constant current through said reservoir and the skin of the patient.

71. A medicament patch as recited in claim 70, wherein said electric circuit is coupled through said reservoir to the skin of a patient.

72. A medicament patch as recited in claim 70, wherein said electric circuit is coupled through the skin of a patient to said reservoir.

73. A medicament patch as recited in claim 71, wherein said electric circuit comprises a field effect transistor source-to-drain series-connected between said power source and the skin of a patient.

74. A medicament patch as recited in claim 73, wherein the gate of said field effect transistor is coupled through a tuning resistor to said power source.

75. A medicament patch as recited in claim 73, wherein the gate of said field effect transistor is coupled to a dedicated power source.

76. A medicament patch as recited in claim 70, wherein said electric circuit comprises:
(a) an operational amplifier; and
(b) amplifier output current stabilization means for maintaining the delivery of said substantially constant current by said operational amplifier through said reservoir and the skin of a patient.

77. A medicament patch as recited in claim 72, wherein said electric circuit comprises:
(a) an operational amplifier; and
(b) amplifier output current stabilization means for maintaining the delivery of said substantially constant current by said operational amplifier through said reservoir and the skin of a patient.

78. A medicament patch as recited in claim 77, wherein said output current stabilization means comprises a feedback loop between a negative input terminal of said operational amplifier and said medicament reservoir.

79. A medicament patch as recited in claim 78, wherein said feedback loop comprises a branch of a voltage divider coupling the ground for said electric circuit to said medicament reservoir.

80. A medicament patch as recited in claim 79, wherein said electric circuit further comprises voltage reference means for providing a substantially invariant voltage to a positive input terminal of said operational amplifier.

81. A medicament patch as recited in claim 80, wherein said voltage reference means comprises:
(a) a zener diode coupled between said positive input terminal of said operational amplifier and a ground for said electric circuit; and
(b) a bias resistor coupled between said power source and said positive input terminal of said operational amplifier.

82. A medicament patch as recited in claim 80, wherein said electric circuit further compnses:
(a) a voltage regulator coupled between said power source and said positive input terminal of said operational amplifier; and
(b) regulator output voltage stabilization means for maintaining the delivery of a substantially constant voltage by said voltage regulator to said positive input terminal of said operational amplifier.

83. A medicament patch as recited in claim 82, wherein said regulator output voltage stabilization means comprises a feedback loop between said positive input terminal of said operational amplifier and an auxiliary input terminal of said voltage regulator.

84. A medicament patch as recited in claim 83, wherein said feedback loop comprises:
(a) a feedback resistor coupled between the output terminal of said voltage regulator and said positive input terminal of said operational amplifier; and
(b) a feedback signal line coupled between said positive input terminal of said operational amplifier and said auxiliary input terminal of said voltage regulator.
